(19) United States
(12) Patent Application Publication
(10) Pub. No.: US 2013/0138110 A1
(43) Pub. Date: May 30, 2013

(54) DEVICES FOR ADAPTING BONE

(71) Applicant: Adam Hacking, Somerville, MA (US)

(72) Inventor: Adam Hacking, Somerville, MA (US)

(73) Assignee: Adam Hacking, Somerville, MA (US)

(21) Appl. No.: 13/813,998

(22) PCT Filed: Aug. 2, 2011
(86) PCT No.: PCT/CA2011/000896

§ 371 Date: Feb. 4, 2013

Related U.S. Application Data
(60) Provisional application No. 61/371,186, filed on Aug. 6, 2010.

(51) Int. Cl. A61B 17/16

(52) U.S. Cl. CPC A61B 17/1659
USPC 606/84

(57) ABSTRACT

A device for adapting a surface roughness of bone, the device comprising: a hollow body having a delivery tip at a distal end, the body being removeably attachable to a handle; a means for supplying fluid to and from the delivery tip to impart a surface roughness similar to that of a bone fracture surface. A device for adapting a surface roughness of bone, the device comprising: a body having an impacting tip at a distal end, the body or the impacting tip being removeably attachable to a handle; a means for generating reciprocating motion of the impacting tip, wherein the impacting tip is arranged to impart a surface roughness similar to that of a bone fracture surface.

### FIELD OF THE INVENTION

The invention relates generally to devices for adapting bone including bone grafts, methods for adapting bone, use of the devices for adapting bone, and bone grafts adapted according to the methods and devices of the invention, all for promoting bone growth and regeneration.

### BACKGROUND OF THE INVENTION

Damage to bone can occur through fracture, injury, disease or surgery and may affect any part of the skeletal system. Bone grafts are often used to assist in the repair or healing of damaged bone, for example in the fields of orthopaedics, maxillo-craniofacial, and periodontics.

However, the use of bone grafts to repair bone has a number of disadvantages such as a relatively high failure rate due to low osteoinductivity of the bone graft, the need for adjunct therapies, and donor site morbidity.

More recently, it has been discovered that adapting, such as by texturing, either the damaged bone (host) or the bone graft may stimulate bone regeneration at that site (WO 2009/046517). In WO 2009/046517 filed Oct. 12, 2007, the contents of which are herein incorporated by reference, methods and devices are described for adapting a bone surface to have a surface roughness similar to that of a fracture surface of bone.

It is desired to improve on these methods and devices for adapting bone in order to overcome or reduce at least some of the above described problems.

### SUMMARY OF THE INVENTION

The embodiments of the present invention reduce the aforesaid difficulties and disadvantages.

As described in WO 2009/046517, the contents of which are herein incorporated by reference, the Applicant had made a surprising discovery that some of the physical characteristics (e.g. the morphology, mean centerline roughness (Ra), the microstructures, the macrostructures, and the peak to peak spacings) of the fracture surfaces of bone can stimulate bone regeneration at that site. The Applicant also surprisingly demonstrated that this bone regeneration was predominantly due to the physical surface characteristics and not primarily as a result of the chemical composition of bone. Thereby, bone material surfaces having some or all of these physical characteristics can be used to treat bone damage such as bone fractures or bone defects. The Applicant discovered that this can be achieved by adapting a surface of a bone material (e.g. cortical, cancellous, allograft) such as by selectively removing bone material from the surface. This is surprising, given the general teaching in the fields of orthopaedics and dentistry which is against the removal of the periosteum (tissue covering bone) or the adaptation of bone surfaces.

The Applicant identified the macrostructures of bone fracture surfaces as including peaks having a peak-to-peak spacing which is substantially less than that of an unfractured bone surface. Preferably, the peak-to-peak spacing is less than about 180 μm. More preferably, the peak-to-peak spacing is between about 0.1 and about 180 μm, about 0.1 to 30 μm, about 0.5 to 30 μm or about 0.5 to 20 μm. Preferably, the peaks are randomly distributed across the bone surface. The surface roughness of the fractured bone, as defined by Ra, is more than about 0.1 μm. More preferably, the Ra is between about 0.1 to 400 μm, about 0.5 to 400 μm, about 0.1 to 20 μm or about 0.5 to 20 μm.

From a first aspect, there is provided a device to impart a surface roughness similar to that of the surface of fractured bone, as defined above and in WO 2009/046517. The device is arranged to be a minimally invasive surgical (MIS) device. A MIS device has reduced tissue disruption, reduced patient discomfort and faster recovery. The MIS device of embodiments of the present invention require only a very small incision. Other manipulating tools and devices are not required. Embodiments of the device of the present invention can function effectively in restricted spaces such as the intramedullary canal, the space within long bones.

Embodiments of the device of the present invention can impart a suitable surface roughness to bone surfaces by the use of suitable impact tips, or by etching, or by applying bioactive particles or blasting particles, or by combinations of the aforementioned. Other embodiments of the devices are arranged to deliver liquid at a suitable pressure for adapting a surface of bone. These devices can be arranged to deliver the liquid in bursts and thin streams (e.g. micron scale diameter). Further embodiments of the devices include a laser which can create micron-sized pits and cavities in a bone surface. Devices which combine any of these surface adapting techniques are also included within the scope of the present invention.

Embodiments of the device are suitable for roughening cortical and cancellous bone surfaces (both in vivo and in graft form). Cortical bone forms the outer wall of most long bones, while cancellous bone often fills space between the cortical bone. Cortical bone is relatively dense and non-porous and the surface morphology is not optimal for new bone formation and/or graft integration. Cancellous bone is comprised mainly of interconnected pores which are enclosed by struts of dense bone, which like cortical bone, possess a smooth surface texture. This surface morphology is not optimal for new bone formation and graft integration.

The external surface of cortical bone grafts interact and ideally bond with the surrounding tissue. Because these grafts are dense, there is very limited interaction with the internal volume. In contrast, the internal surface of cancellous bone grafts often interact with the surrounding tissue. In this regard, it has long been recognized that tissue will grow within porous structures and within porous bone. This is important since cancellous bone is often used as a void filler and the desired host tissue response is growth within the porous structure and integration into the host tissue.

Bone forming cells are called osteoblasts. Osteoblasts grow and produce a mineralized matrix on surfaces such as bone and it is this interaction with grafts that leads to successful skeletal integration. The addition of the above identified surface texture to bone is effective in stimulating new bone formation to a much greater extent than untextured, ‘native’ surfaces similar to those found on both cortical bone and cancellous struts.

Embodiments of the device of the present invention can successfully texture both cortical and cancellous bone. In the past, known mechanical impact methods were found to be inefficient for texturing the large sequestered surface area within porous materials such as cancellous bone. Attempts to texture these materials using traditional line-of-sight techniques, such as particulate blasting, laser ablation or plasma spray based processes could not access the internal surfaces sequestered within the material or generally obscured or clogged the pores, preventing tissue ingress and graft incorporation.

By means of embodiments of the invention, the treatment of damaged bone such as bone fracture and defects will be easier and cheaper. For example, the method of the invention provides an adapted bone surface for promoting bone regeneration which is simple, effective and not damaging to the structural integrity of the bone material itself. Advantageously, the surface can be adapted (textured) without the use of complex apparatus or devices.

The embodiments of the device of the invention are envisaged to have application in various cranio-facial, maxillo-facial and orthopedic procedures such as treating long bone fracture, mal-union, non-union surgical procedures, joint fusion, fractures and compression of cancellous bone and bone defect repair including defects from surgical incisions and disease for preservation or repair of the bone, and implant fixation, for example.

In one embodiment of the device, bone may be etched intra-operatively by exposure to acid or other etching solutions. An etching solution may be applied to the bone surface using a brush, mist or sponge or a gel-like material (solid that will not flow). However it may be beneficial from a clinical standpoint to limit the extent and exposure of the bone and bone marrow to the etching solution. One embodiment of the device of the present invention constrains the application of the acid. The device may also apply, wash and remove the acid from the bone surface. Also, a gel may be first injected as a barrier to acid infiltration into the cancellous bone. The etching solution can be delivered under pressure to drive the gel back exposing the adjacent bone and preventing deep penetration of the etching solution. After a brief etch period, the etching solution and the gel can be collected by suction and repeated washing.

In another embodiment of the device, cancellous or cortical bone may be textured by contact with a device having a contacting tip consisting of many fibers (metal or plastic, e.g. nylon). The fibers may have tips that are adapted for contacting the bone, such as coated with a bonded hard particle, ceramic coating or a hardened metal. The fibers can be used to access a portion of the surface of cancellous bone within the porous spaces. Fibers may vibrate in many directions at both ultrasonic and non-ultrasonic frequencies. Fibers may move in micron and millimeter distances in all directions.

In another embodiment of the device, a slurry of bone particles can be delivered into the porous space of the cancellous bone to further enhance bone response. The bone particles may have a textured surface obtained by grinding or crushing bone, by brief etching or by precipitation.

The above devices and methods are also applicable to the adaption of cortical bone. Compared to mechanical treatment of the bone surface, immersion in a solution may provide a faster and more uniform method to apply a texture to cortical bone.

From another aspect, there is also provided methods of adapting a surface of bone including using the devices of the present invention.

### DEFINITIONS

As used herein, the term “bone” includes the whole or any part of natural bone anywhere in a body of an animal, such as a human.

As used herein, the term “bone graft” includes the whole or any part of a graft derived from natural bone, such as cortical or cancellous bone. The term includes autografts, allografts or xenografts which may or may not be fully or partially demineralized.

As used herein, the term “bone material” includes any or all of the material making up bone, such as bone mineral matrix and intercellular bone tissue substance.

As used herein, the term “host bone” refers to a bone site in an intended recipient (host) of a bone graft. The bone site may be near or at a bone fracture, bone defect, bone cut or any other type of bone damage.

As used herein, the term “bone fracture surface” refers to the fracture surface of compact bone between the endosteal or periosteal surfaces.

As used herein, the terms “macroroughness”, “macrotexture” or “macrofeatures” refer to larger surface features within the micron range. These larger surface features include peaks which have smaller surface features (“microfeatures”, “microroughness” or “microtexture”) superimposed thereon.

As used herein, the terms “microroughness”, “microtexture” or “microfeatures” refer to smaller surface features within the micron scale and which are smaller than the macrofeatures. The terms “macrotexture” and “microtexture” are illustrated in FIG. 2 of WO 2009/046517.

The term “texture” or “roughness”, as used herein, is meant to encompass both the microtexture and the macrotexture of a surface within the micron range.

The terms “mean roughness (Rₐ)”, “root-mean-square roughness (R_{q})”, peak-to-peak spacing, peak-to-valley height and peak diameter are parameters for defining the texture or roughness of a surface and are illustrated in FIG. 1 of WO 2009/046517. Mean roughness (Rₐ) is defined as the average deviation from the mean centerline roughness of the surface (macrotexture and microtexture). It is quantified using atomic force microscopy or white light interferometry in ways known to a person skilled in the art.

The root-mean-square roughness (R_{q}) is defined as the root-mean-square deviation of the profile from the mean line over one sampling length. The peak-to-peak spacing is defined as the shortest distance between adjacent peaks as manually measured from SEM photographs of the surface. The peak-to-valley height is defined as the distance from a base of the peak to a tip of peak as manually measured from SEM photographs of the surface. The peak diameter is defined as the longest distance measurable along the tip of a peak as manually measured from SEM photographs of the surface. Since the fracture surface is fractal-like (i.e. comprised of similar repeating features as the measurement scale increases ore decreases), the surface features are scale dependant. This means that at different scales the values of these features may change. Features of interest were quantified using SEM at 1000-5000× mag.

The phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements. In the drawings, like reference characters designate like or similar parts.

### BRIEF DESCRIPTION OF THE DRAWINGS

Further aspects and advantages of the present invention will become better understood with reference to the description in association with the following drawings in which:

FIGS. 1, 2 and 5 illustrate a first embodiment of a device of the present invention;

FIGS. 3 and 4 illustrate a first embodiment of an impact tip for use with the device of FIGS. 1 and 2;

FIG. 6 illustrates results from Example 1 using the device of FIGS. 1, 2 and 5;

FIGS. 7 and 8 illustrate a second embodiment of the device of the present invention;

FIGS. 9 and 10 illustrate a third embodiment of the device of the present invention;

FIGS. 11 and 12 illustrate a fourth embodiment of the device of the present invention;

FIGS. 13, 14 and 15 illustrate a fifth embodiment of the device of the present invention;

FIGS. 16, 17 and 18 illustrate a sixth embodiment of the device of the present invention;

FIGS. 19, 20 and 21 illustrate a seventh embodiment of the device of the present invention;

FIGS. 22, 23 and 24 illustrate an eighth embodiment of the device of the present invention;

FIGS. 25, 26 and 27 illustrate a ninth embodiment of the device of the present invention;

FIG. 28 illustrates Example 3;

FIGS. 29 and 30 illustrate Example 4;

FIG. 31 illustrates Example 5; and

FIG. 32 illustrates Example 6.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways.

Briefly, an aspect of the invention comprises a device 10 for adapting a surface of a bone material. Preferably, the surface of the bone material is adapted to have a surface roughness similar to a surface roughness of a bone fracture surface.

The surface roughness, within the micron range, preferably comprises macrofeatures (a primary structure), also referred to as peaks, and microfeatures (a secondary structure). The microfeatures are smaller than the macrofeatures and are applied to, or superimposed on, the surface of the macrofeatures. The peaks are arranged or distributed across the surface in a random or un-orientated manner. In other words, the arrangement of the peaks does not form a regular pattern such as striations. The average peak-to-peak spacing is substantially less than that of smooth surfaces of bone. Preferably, the average peak-to-peak spacing of the surface is less than about 180 μm. More preferably, the average peak-to-peak spacing of the surface is less than about 30 μm, between about 0.1 and 30 μm, between about 0.5 and 30 μm, or between about 0.5 and 20 μm. The average peak-to-valley height is substantially more than that of smooth surfaces of bone. The average peak-to-valley height is preferably more than about 1 μm. More preferably, the average peak-to-valley height is between about 1 and 15 μm, about 1 and 10 μm, or about 1 and 5 μm. The average peak diameter is less than about 140 μm. More preferably, the average peak diameter is between about 0.1 and 20 μm, about 0.1 and 15 μm, or about 0.1 and 10 μm.

The surface has a roughness, as defined by Rₐ, which is substantially more than that of smooth (unfractured) bone. Preferably, the Rₐ is more than about 0.1 μm. More preferably, the Rₐ is between about 0.1 to 400 μm, about 0.5 to 400 μm, about 0.1 to 20 μm or about 0.5 to 20 μm.

The bone material can be a bone graft or a bone within the body of a patient (in vivo) such as a cortical bone or a cancellous bone. The bone surface can be an inner or an outer surface of the bone such as the endosteal and periosteal surfaces of bone.

The bone surface to be adapted may be the outer cortical surface of bone. For example, the cortical or periosteal surface around or adjacent to a fracture can be adapted as described, prior to applying a graft or other fixation means across the fracture. The roughened periosteal surface will have the effect of encouraging bone formation and regeneration around the bone fracture site and improved fixation of the graft or other fixation means across the fracture. The applied bone graft may also have a roughened surface contactable with the roughened host bone surface to further enhance bone formation at the interface of the bone graft and the host bone surface.

The bone surface to be adapted may include the inner endosteal surface of bone. For example, in the case of a patient undergoing a hip replacement, a metal implant is usually placed into the femoral canal and may be fixed in place by bone cement. The use of bone cement may be avoided and the fixation of the implant to the inner cortical bone may be improved by applying the method of the present invention to at least a portion of the bone surface in the femoral canal (endosteal surface) to promote bone regeneration at that surface.

The bone surface to be adapted may be an intermediate bone surface such as a cut surface of cortical bone which may have been cut during a re-section or excision e.g. to remove a tumour or infection.

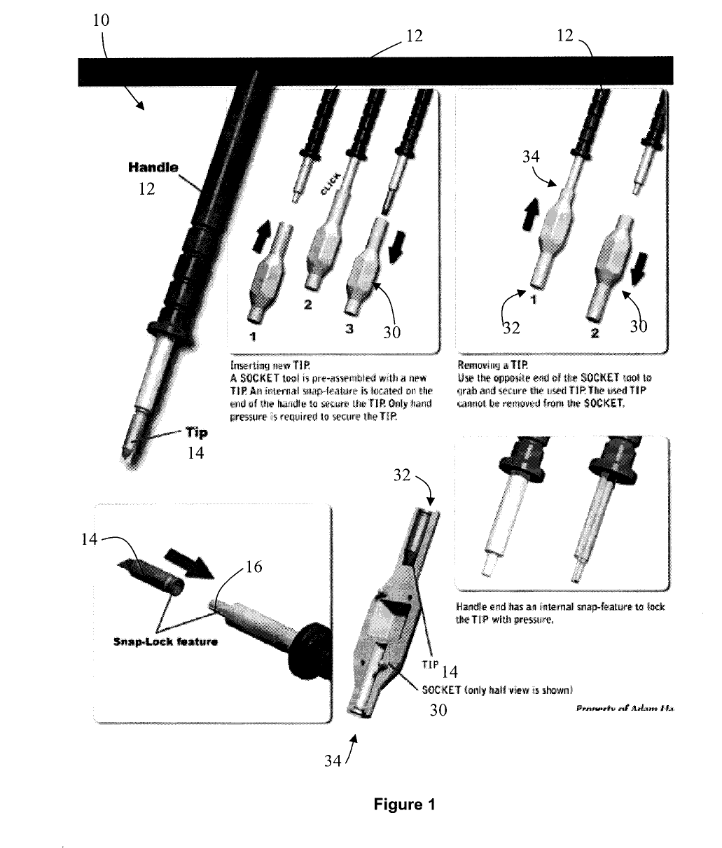

A first embodiment of the device 10 is illustrated in FIGS. 1 to 4 and comprises a body having a handle 12 at a first end, and a second end (“distal end”) which is arranged to receive an impact tip (“tip”) 14 for imparting the desired surface roughness to the bone. The impact tip 14 is arranged to be removeably attachable to the second end. The impact tip 14 can be disposable or reusable. The device 10 is arranged so that the impact tip 14, once assembled on the body, can mechanically reciprocate (e.g. move backwards and forwards) relative to the handle or body to repeatedly impact a surface which it is held against. In this regard, the entire device 10 may actuate or just the second end with the impact tip 14 attached. The handle 12 and the impact tip 14 may not be aligned as illustrated in FIG. 1 but may be angled in relation to each other. In fact, the second end of the device may be moveable with respect to the handle to provide different accessibility of the impact tip. The handle can be made of plastic or the like and can be of an ergonomic shape and size to facilitate the holding of the device by a user and for guiding the tip. The handle may be provided with a control button (not shown) to turn actuation of the impact tip on and off. Alternatively, the control button may be provided externally of the device.

The mechanism (not shown) to provide the reciprocating movement of the impact tip can be partially or completely contained within the handle, or can be external to the device 10, and is arranged in a manner that would be clear to persons skilled in the art. For example, in one embodiment, the mechanism includes a motor and a control unit for controlling the amplitude and frequency of motion. The range of motion of the impact tip is microns to millimetres, preferably microns, for the amplitude and hertz to kilohertz, preferably kilohertz, for the frequency. Motion can be tuned to generate a non-striated surface. The mechanism may be powered by battery, compressed air, through the mains, or the like. The mechanism may include a magneto-restrictive or a piezo-electric device.

The second end of the device is sized and shaped to be received into a hollow body of the impact tip 14. Preferably, the second end of the device is generally cylindrical shaped and has a locking means for fixing the impact tip to the second end. As illustrated in FIG. 1, the locking means can be a raised tongue 16 on the second end which is received into the hollow body of the impact tip 14 to snap fit the impact tip onto the second end. The tongue may be resilient and made of a plastic or a metal.

The internal surface of the impact tip and the external surface of the device second end have a mis-matching (i.e. Morse) taper which avoids or reduces movement between the impact tip and the device second end. In fact, the impact tip 14 can self tighten on the device second end with each impact. In one embodiment, the device second end has circumferential lines (such as those formed by machining) which can also aid anchorage of the impact tip14. The fit of the impact tip over the device second end is preferably very tight once installed.

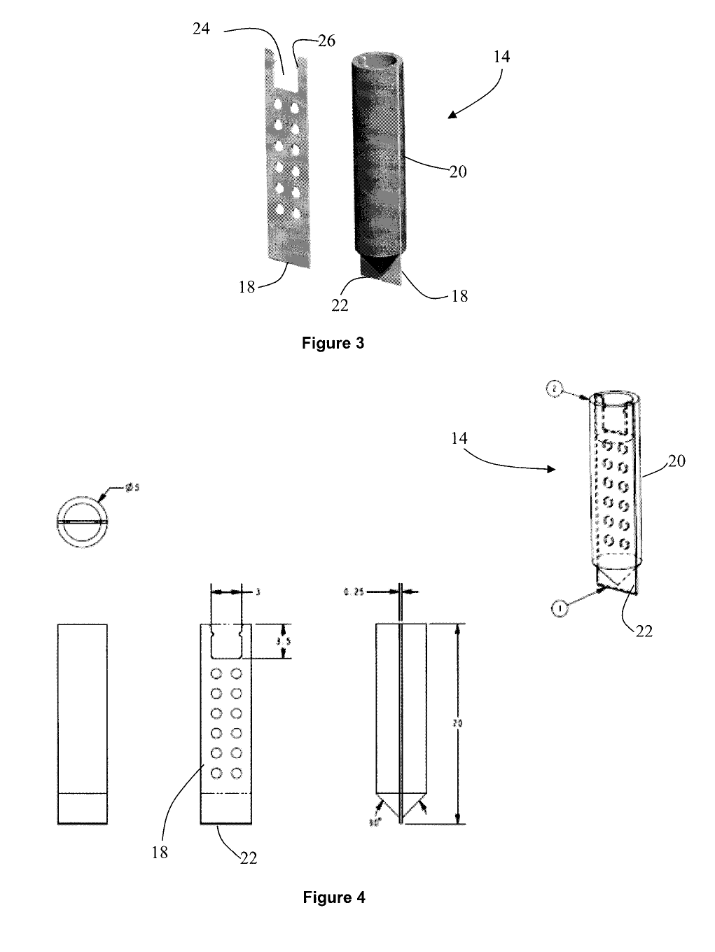

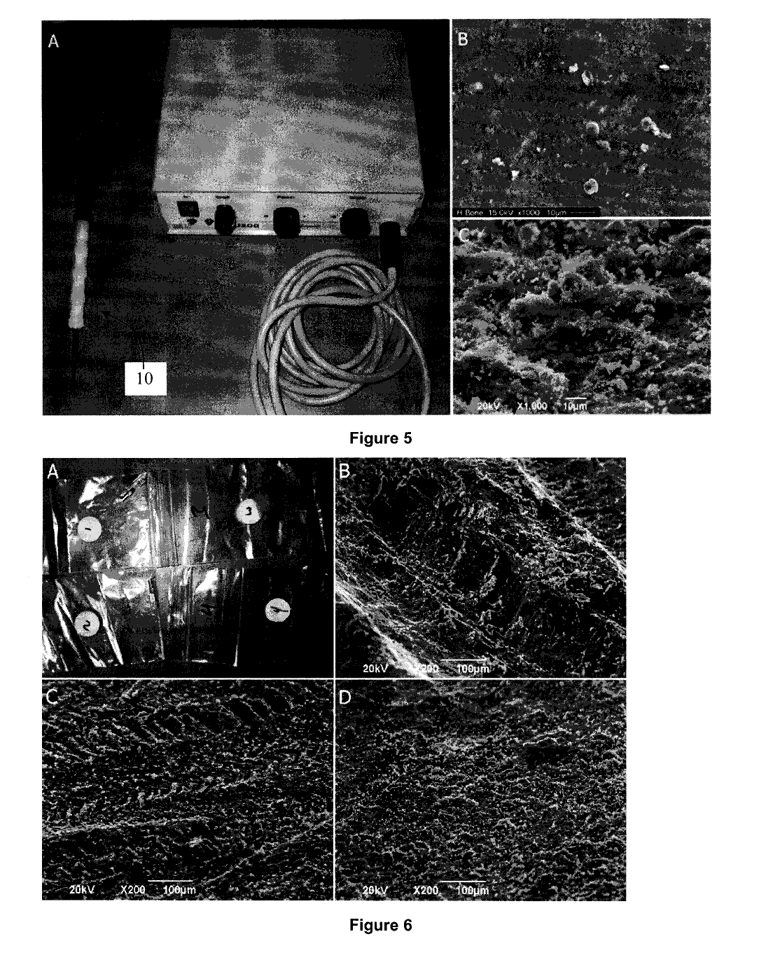

The impact tip 14 is illustrated in FIGS. 3 and 4 and comprises a blade 18 and a holder 20. In the embodiment of FIGS. 3 and 4, the blade 18 is an elongate metal sheet having a cutting edge 22 at one end for contacting the bone surface. At the other end, the metal sheet is recessed 24 to receive the second end of the device. The recess 24 has a raised ring of material 26 for assisting in retaining the impact tip 14 on the device second end once assembled. A body of the blade may have openings for reducing the weight of the blade without compromising its strength. The cutting edge 22 may be scalloped (1-30 μm width, 1-50 μm depth). The cutting edge may be formed by laser cutting, photo etching or any other suitable method. The cutting edge 22 may also be textured, such as by grit blasting or diamond coating. It will be appreciated that the cutting edge of the impact tip can take any form or shape necessary to impart the desired surface roughness on bone. There may also be a plurality of cutting edges (e.g. in stacks or layers) so that each impact tip has multiple contact surfaces. The plurality of cutting edges may be offset (i.e. not parallel to one another) to avoid creating regular striations. The holder 20 is a plastic sleeve for receiving the blade 18 such that the blade is encased by the holder except for the cutting edge which extends from an end of the holder 20. Preferably, the holder 20 has a hollow cylindrical body made by injection moulding, or any other suitable method. The holder body may be made as two half-pipes which are sandwiched around the blade. Advantageously, the blade 18 and holder 20 construction of embodiments of the present invention results in a light, strong, safe and low cost impact tip for adapting bone surfaces. The second end of the device and an internal surface of the impact tip 14 may have a matching taper to assist in the retaining of the impact tip on the second end of the device.

The device 10 may also include a tool 30 for assembling the impact tip 14 on the device second end without the user having to touch the impact tip 14, and for removing the impact tip from the device second end without the user having to touch the impact tip (FIG. 1). The tool (“socket tool”) 30 comprises a body having a first end 32 for receiving a new unused impact tip for assembly on the device and a second end 34 for receiving a used impact tip 14 and removing it from the device 10.

In use, a new impact tip 14 is placed into the tool first end 32 and the tool first end is positioned over the device second end so that the impact tip is brought into engagement with the device second end. The tool is pushed up against the device second end to snap (‘push-fit’) the impact tip over the device second end. A ‘click’ may be heard when the impact tip is positioned correctly. Once the impact tip is retained on the device second end, the tool can then be pulled away from the device. In this way, the impact tip can remain sterile during assembly of the device and the impact tip.

The impact tip is removed from the device second end by inserting the impact tip into the second end of the tool. The socket grabs the tool and provides more resistance to dislocation than the initial snap fit. The impact tip can then be disposed of safely.

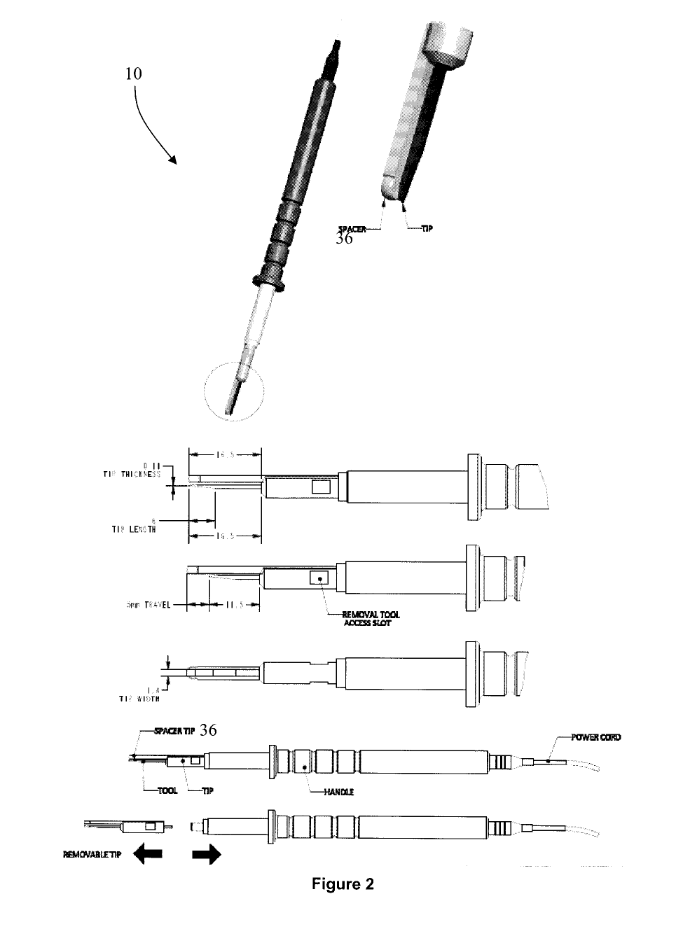

The device may also include a spacer means 36 (see FIG. 2) to maintain the impact tip 14 at a distance from the bone surface. The spacer means is attached to the non-moving part of the device (either the body or the handle) to enable the tip to maintain only intermittent contact with the surface of the bone. The spacer means 36 is located adjacent the impact tip 14 and permits movement of the tip as the user applies pressure to position the tip against the bone. The dimensions given in FIG. 2 relating to the spacer means and translation of the tip 14 are relevant for the mechanical actuation of the tip when used with an electric motor. When movement of the tip 14 is driven by ultrasonic means, the translation of the tip 14 is expected to be less. The size of the spacer means 36 is selected according to the size and shape of the impact tip such that when the spacer means is pressed against the bone surface, the impact tip should be able to contact and depart the bone surface.

In summary, the device 10 of FIGS. 1 to 4 is for applying a texture or surface roughness to bone where open access to the bone surface is available, for example on bone grafts or on exterior surfaces of bone. The device 10 can be held like a pencil against the bone surface to apply a surface roughness to the bone. The impact tip 14 is made to move backwards and forwards against the bone surface to impart the surface texture i.e. up and down generally perpendicular to the bone surface.

### Example 1

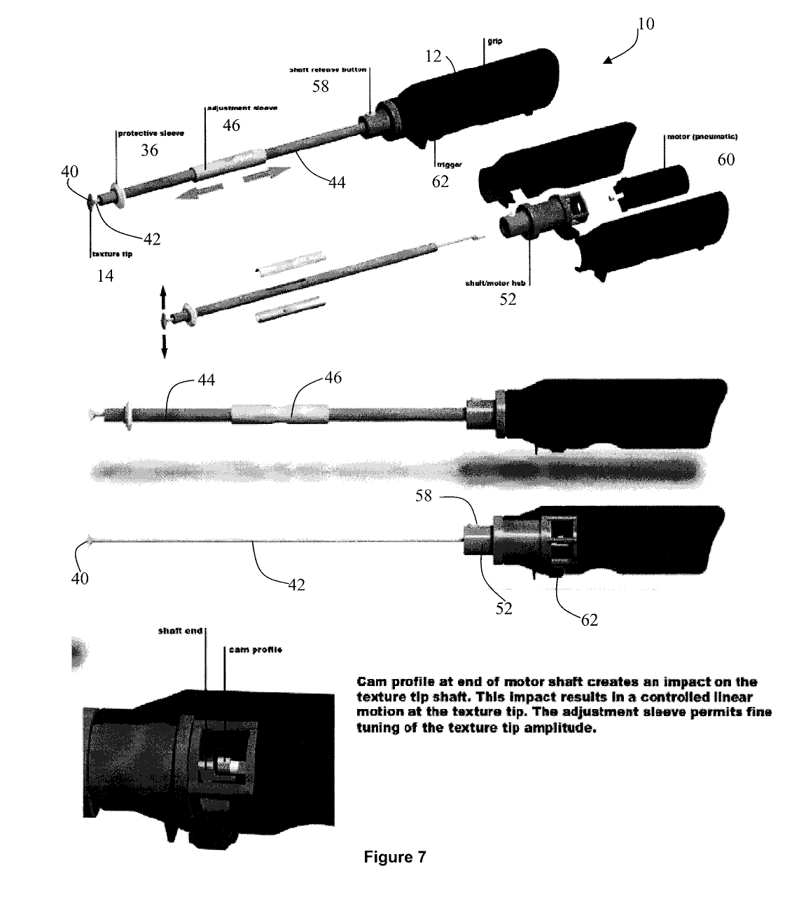

FIG. 5A illustrates an embodiment of the device of FIGS. 1 to 4. There is provided a control unit capable of reciprocating the tip at a rate of about 30 kHz and an amplitude of about 20 μm. Peak height and peak spacing on the tip were 10 μm. FIG. 5B is an SEM image of relatively smooth endosteal bone surface (Rₐ-0.6 μm) with red blood cells visible, before adapting with the device of the present invention. FIG. 5C illustrates a cortical bone surface which has been adapted using the device of FIG. 5A.

### Example 2

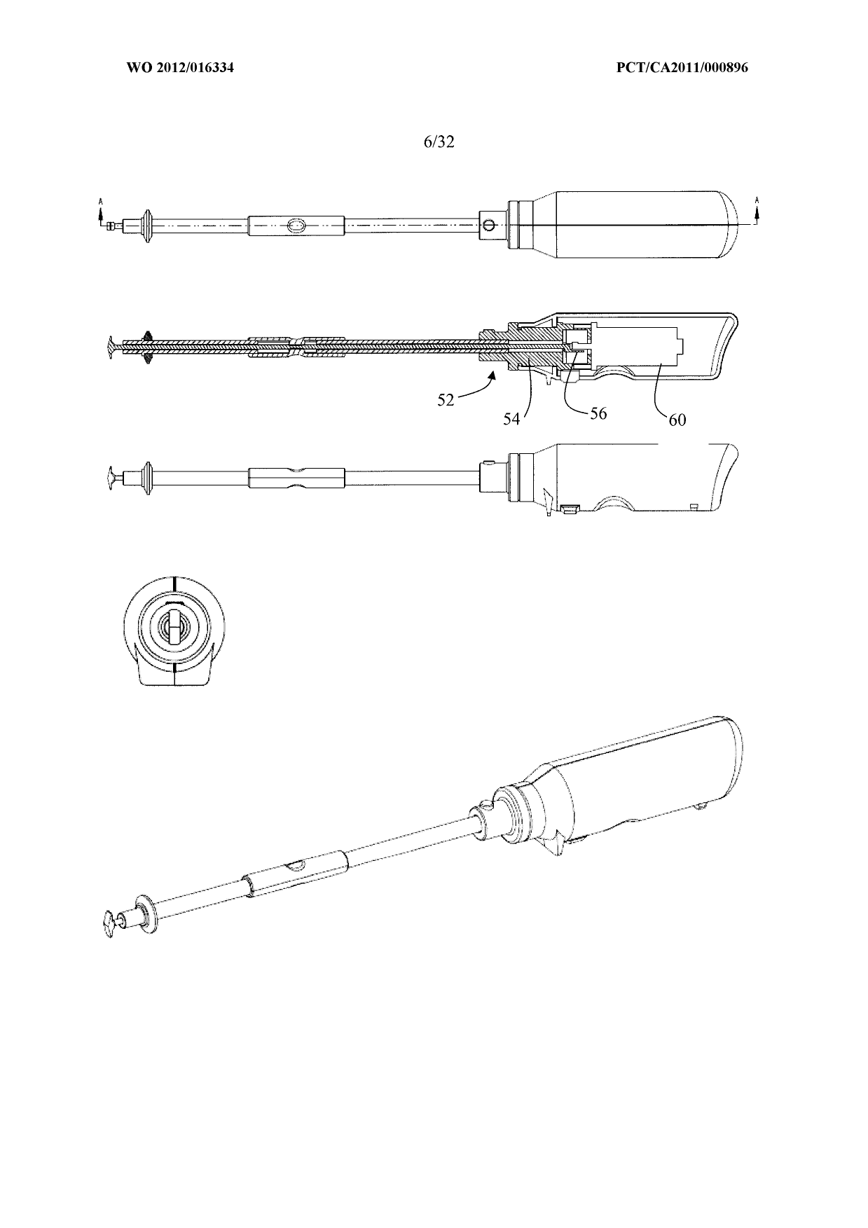

Experiments were conducted on a cortical bone surface to optimize the impact tip geometry for the device of FIGS. 1 to 4, as well as for other embodiments of the device. Portions of the impact tips were diamond coated with different diamond sizes ranging from 500-10 μm diameter (FIG. 6A) in order to evaluate different surface roughnesses. Reciprocating movement was 30 kHz and tip translation was 20 μm. FIGS. 6B, 6C and 6D show the optimization of the tip geometry for optimised bone cell growth on polished bone surfaces. For any combination of frequency and amplitude the desired outcome is not a striated structure, but an irregular surface. It was found that a striated surface may be made more irregular by using the spacer means 36 (see FIG. 2). FIG. 6B shows a striated surface created by texturing with a tip coated with large diamond particles (about 500 μm diameter). FIG. 6C shows a moderately striated surface created by texturing with a tip coated with moderately sized diamond particles (about 100 μm diameter). FIG. 6D shows uniformly and irregularly textured surface created by texturing with a tip coated with small diamond particles (about 25 μm diameter). It was found that particles larger than the translation of the tip (about 25 μm) left striations on the bone surface. Sizes less than about 25 μm diameter were found to result in an irregularly textured surface without striations (FIG. 6D). It was considered that striated and textured surfaces shown in FIGS. 6B and 6C may be optimal for soft tissue attachment. Thus optimal tip features are dependent upon tip amplitude and generally the peak to valley height should be less than the amplitude.

The spacer means 36 is located near the impact tip 14 of the device and attached to the non-moving part of the device 10 to enable the tip to maintain only intermittent contact with the surface of the bone. The optimal surface roughness of the impact tip was found to be about 5 μm peak to valley spacing, 10 μm peak to peak spacing and less than 1 μm peak diameter. It should be noted that the optimal roughness varies with the impact tip frequency and amplitude and can be “tuned” to produce an irregular and not striated surface according to the roughening mechanism used. For example, electric and air driven motors produce lower frequency and higher amplitude than piezo or magneto restrictive devices. As a result the tip geometry can be matched to the motor.

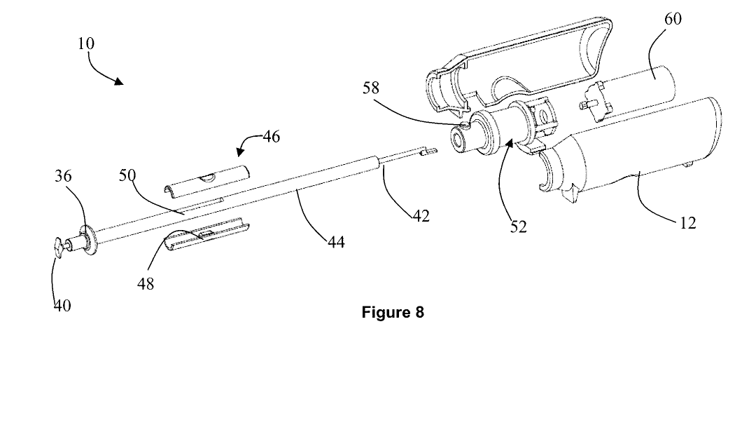

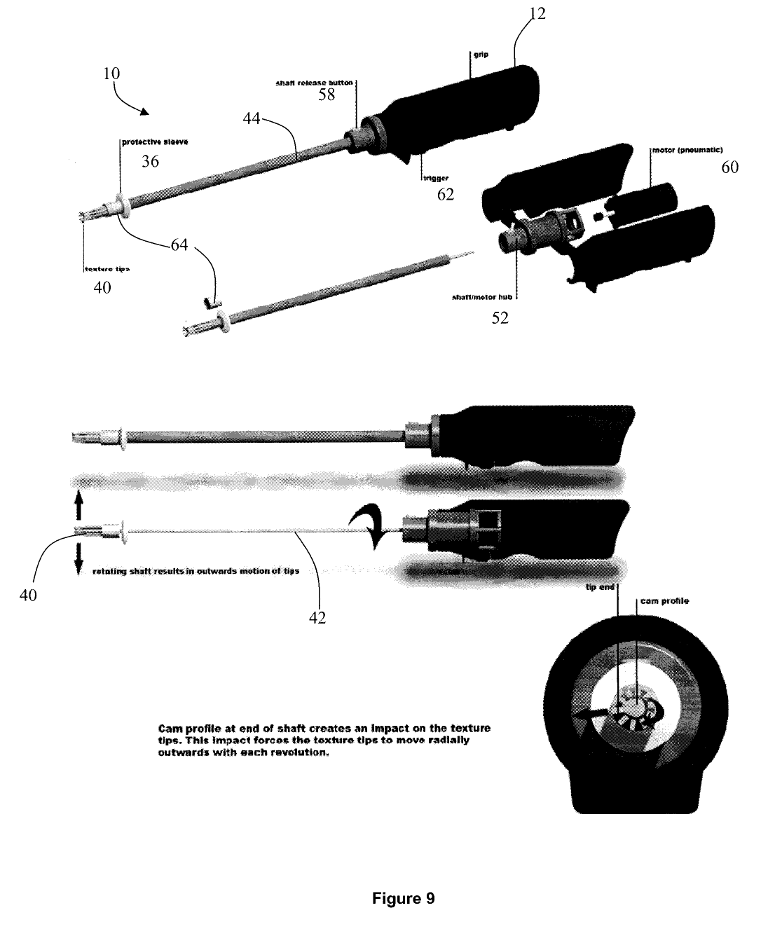

A second embodiment of the device 10 is illustrated in FIGS. 7 and 8. In this embodiment, the device 10 is arranged to be a minimally invasive device for texturing endosteal or periosteal cortices. In this respect, the device 10 of FIGS. 7 and 8 differs from the first embodiment of the device 10 in that the impact tip 14 comprises an impact surface 40 connected to an elongate shaft 42 which is removeably attachable to the second end of the device to connect the impact tip 14 to the handle 12. The impact surface 40 is arranged to move perpendicularly to the handle. The elongate shaft 42 is partially surrounded by a sleeve 44 which has two open ends through which the elongate shaft 42 extends. One end of the sleeve 44 is also received into the second end of the device 10. The sleeve 44 is a protective sheath which is moveable with respect to the elongate shaft 42 of the impact tip 14 to expose varying lengths of the elongate shaft 42 adjacent the impact surface 40. This permits adjustment of the amplitude of motion of the impact surface 40. An adjustment means (‘slider’) 46 is provided on the sleeve 44 for moving the sleeve relative to the elongate shaft 42. As best seen in FIG. 8, the adjustment means 46 has a protrusion 48 which is received in a channel 50 of the sleeve 44. This means that the adjustment means 46 is moveable relative to the sleeve 44 only within the channel 50. Further movement of the adjustment means 46 will adjust the position of the sleeve 44. The adjustment means 46 can also be immoveably attached to the sleeve 44. In this way, the movement of the impact surface can be tuned via one or more pivot points along the elongate shaft 42. Also provided on the sleeve 44 is an annular version of the spacer means 36 of FIG. 2 (“protective sleeve”) for spacing the impact surface 40 from the bone to be textured. The spacer means 36 provides space for the impact tip to move to contact and retract from the bone surface while pressure is put on the device by the user. Otherwise the tip may move and instead of texturing, the device 10 itself moves away from the bone. The spacer means may be resilient. The impact surface 40 is in the form of a shoe or the like having two impact surfaces which are angled with respect to one another. The impact tip can reciprocate backwards and forwards perpendicularly to the elongate shaft 42 and the sleeve 44 to impart a surface roughness in the direction perpendicular to elongate shaft 42. Other shapes and configurations of impact surfaces are also included within the scope of the present invention such as textured surfaces, and multiple or single impact surfaces. The impact surface(s) may be textured to vary its surface roughness such as with a diamond coating or the like.

At the device second end, a receiving unit 52 is provided for receiving the elongate shaft 42 and the sleeve 44 and for removeably connecting them to the handle 12. The receiving unit 52 is attachable to the handle 12 and has an elongate opening 54 for receiving the sleeve 44 and the elongate shaft 42. As the elongate shaft 42 extends further than the sleeve 44, a further opening 56 is provided for receiving the elongate shaft 42 and for imparting the reciprocating movement thereon. A locking mechanism is provided for securing the elongate shaft 42 and sleeve 44 to the receiving unit which may be a snap fit or any other type of fixation mechanism apparent to a skilled person in the art. A release button 58 is provided on the receiving unit 52 for releasing the elongate shaft 42 and sleeve 44 from the receiving unit.

A motor 60 is provided within the handle 12 which is co-operable with the elongate shaft 42 via a gear assembly (not shown) to impart motion to the impact surface 40. The motor 60 initiates a series of standing waves in the elongate shaft 42. The motor 60 may be pneumatic or any other type such as electric (AC/DC) or the like. The motor may rotate and wiggle the shaft 42 by striking it one or more times per rotation with a cam (see FIG. 7). The motor may be piezo- or magneto-restrictive and may simply vibrate the shaft end or tip directly. Control means 62, such as buttons (triggers), may be provided on the handle for turning the motor on and off.

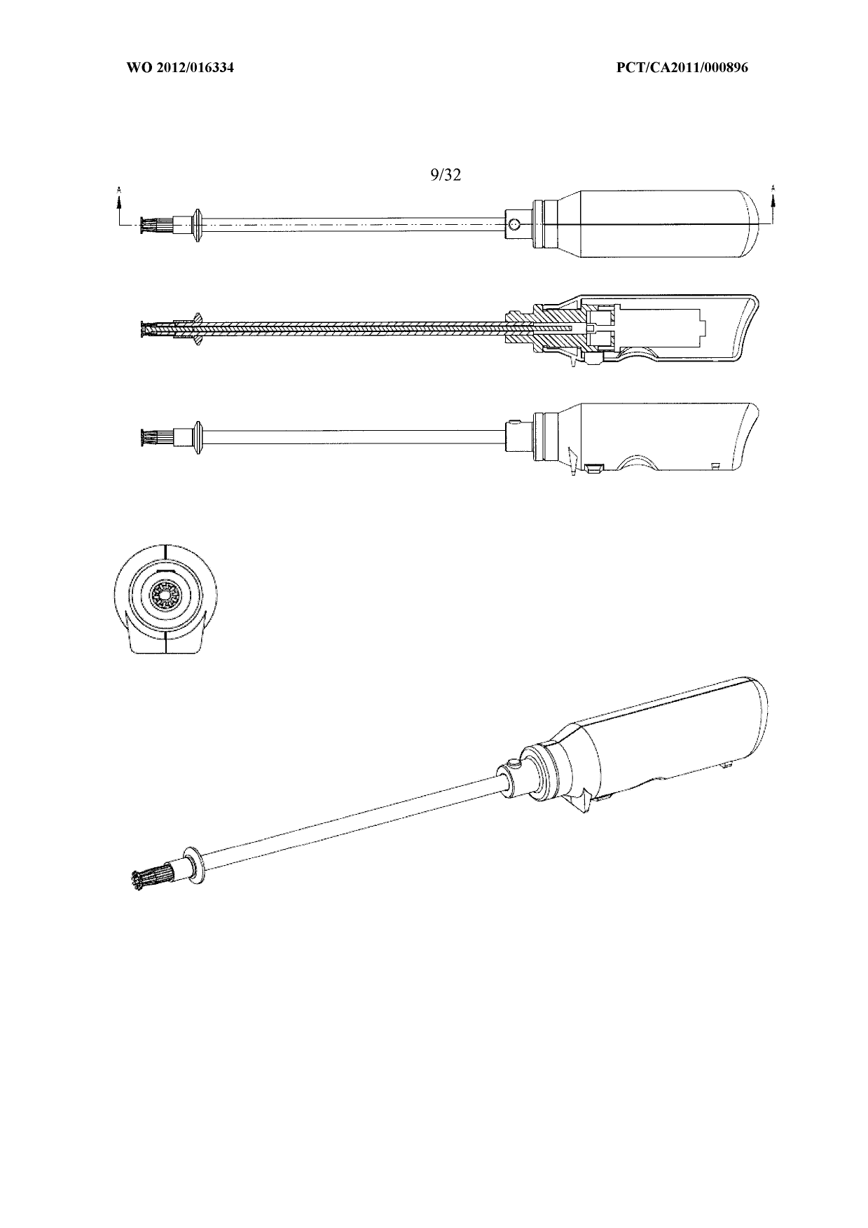

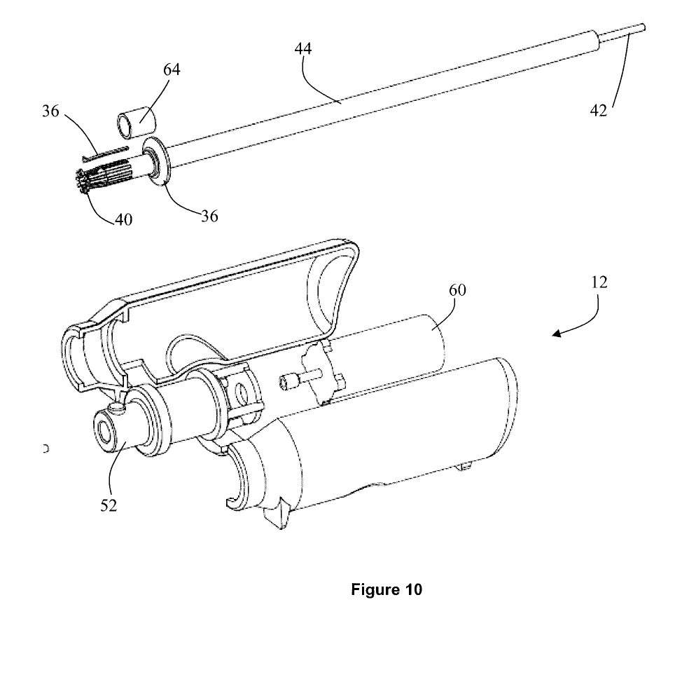

A third embodiment of the device 10 is illustrated in FIGS. 9 and 10 and differs from the device of FIGS. 7 and 8 in that instead of a shoe as an impact surface(s) 40, there are provided a plurality of tips or fingers which outwardly flail as the elongate shaft 42 is made to rotate. Each tip or finger is moveable individually. In this embodiment, the motor 60 can impart a rotational motion, a side-to-side motion (perpendicular to the long axis of the elongate shaft) or a linear motion (parallel to the long axis of the elongate shaft) to the elongate shaft 42. Preferably, the motion is the rotational motion or the linear motion. In other words, the tips or fingers move radially outwards, away from the elongate shaft 42, as the elongate shaft 42 rotates about its long axis. The tips or fingers are resiliently biased to the closed position so that they return to the closed position when the elongate shaft motion is reduced or stopped. In one embodiment, the tips or fingers are cut from a metal or a plastic base. The distal end of the elongate shaft 42 has a cam or protrusion that successively pushes each of the tips/fingers outwardly as the shaft rotates (see FIG. 9). Each finger/tip may be partially or completely textured for imparting the appropriate surface roughness to the bone which it contacts. The outer end of each finger or tip may have a protrusion, such that each finger is like a hammer. By means of any of these configurations, texture can be applied to bone in a direction perpendicular to the elongate shaft.

In this embodiment, the sleeve 44 is not moveable with respect to the elongate shaft 42. In this respect, there is no adjustment means. The sleeve 44 provides rigidity to the elongate shaft so that force and direction can be applied when the tips/fingers are pressed against the bone surface. It also prevents tissue and vessels etc from becoming entangled in the elongate shaft as it moves. A collar 64 is provided which is positioned over the sleeve 44 adjacent the tips and the annular spacer 36. The collar has a similar function to the spacer 36 in that it makes a space from the bone in which the tips can extend and retract while pressure is put on the device by the user. Otherwise the tips may move and instead of texturing, the device itself moves away from the bone. The elongate spacer 36 of FIG. 2 may also be provided (see FIG. 10).

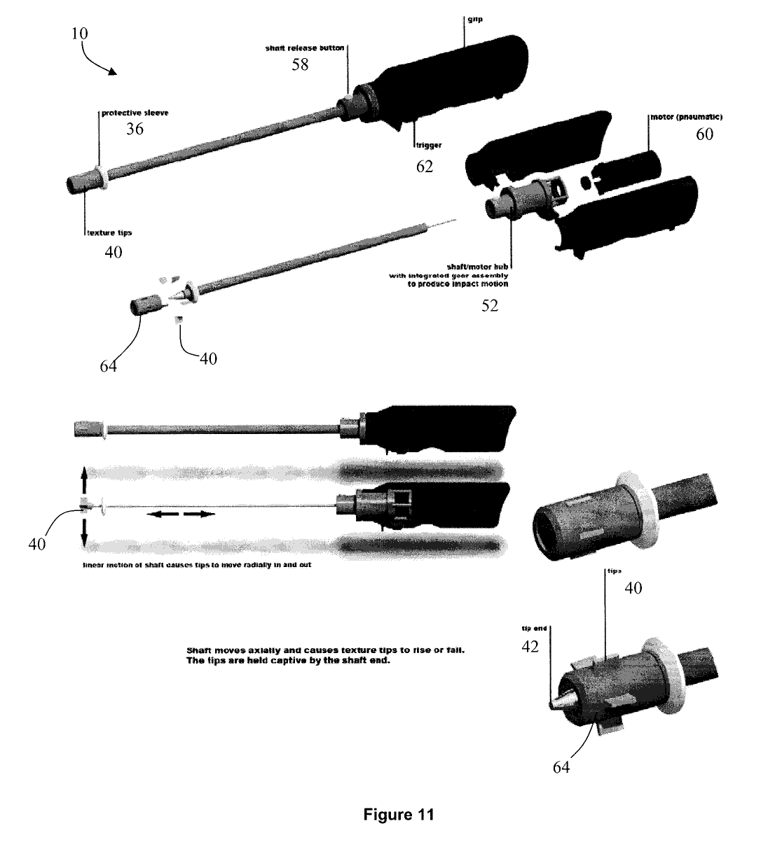

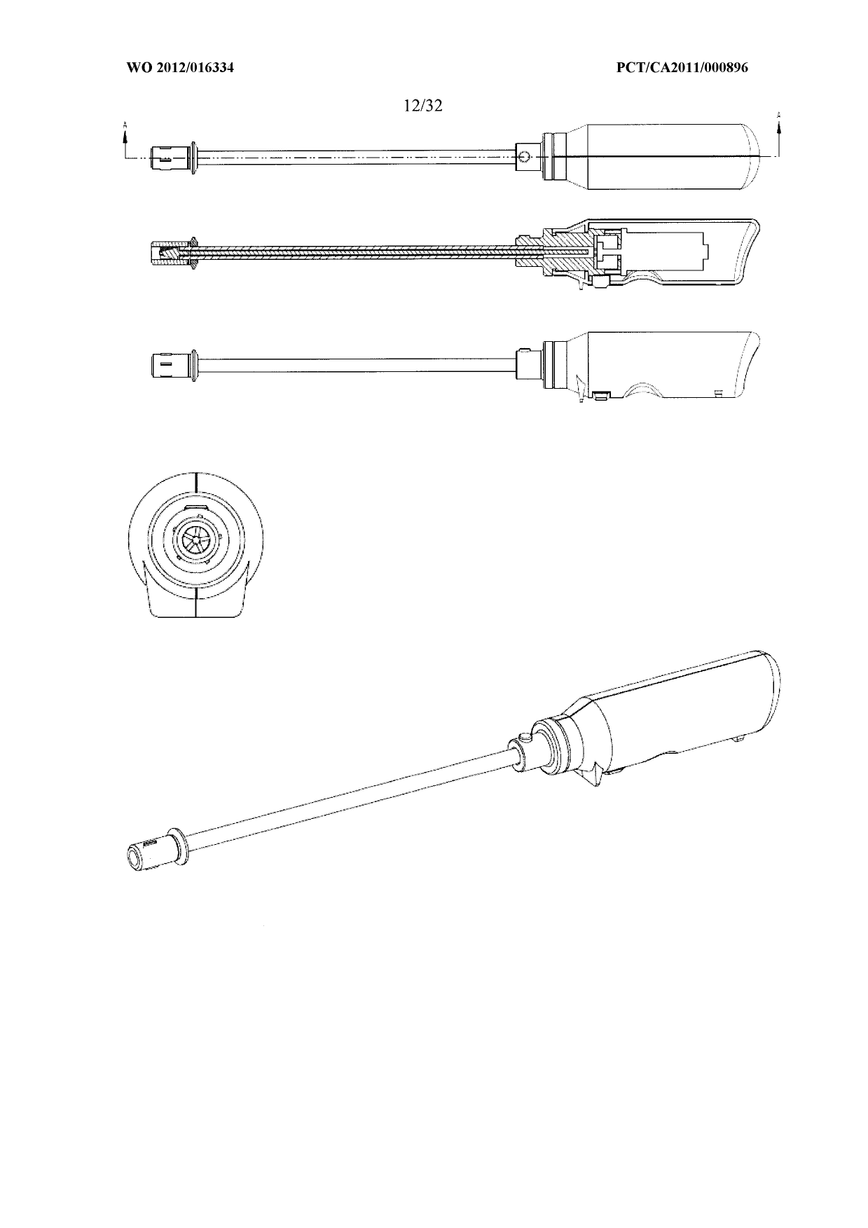

A fourth embodiment of the device is illustrated in FIGS. 11 and 12 and differs from the embodiment of FIGS. 9 and 10 in that the impact surface 40 comprises a plurality of radially extending blades (five blades as illustrated, but can be more or less than five) which extend from the distal end of the elongate shaft 42 and which protrude from corresponding openings in the collar 64. The distal end of the elongate shaft 42 is in the form of an arrow head or the like, which is tapered, such that movement of the elongate shaft parallel to the axis of the elongate shaft 42 causes the taper or the arrow head to push against and withdraw from the blades so that the blades extend and retract from the collar 64. As with the embodiment of FIGS. 9 and 10, there is no adjustment means. This device is particularly well suited to texturing the endosteal cortex. Preferably, the motor 60 imparts a linear motion to the elongate shaft (parallel to the long axis of the elongate shaft) i.e. the elongate shaft moves in and out of the handle 12. The blades move at the same time as they are connected to one another. A spring or other resilient means (not shown) may be provided to return the blades to their original position.

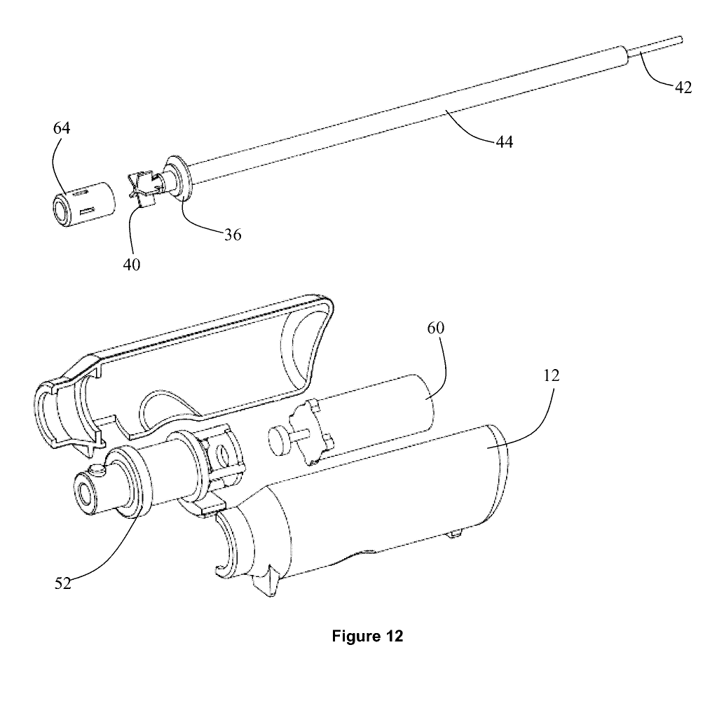

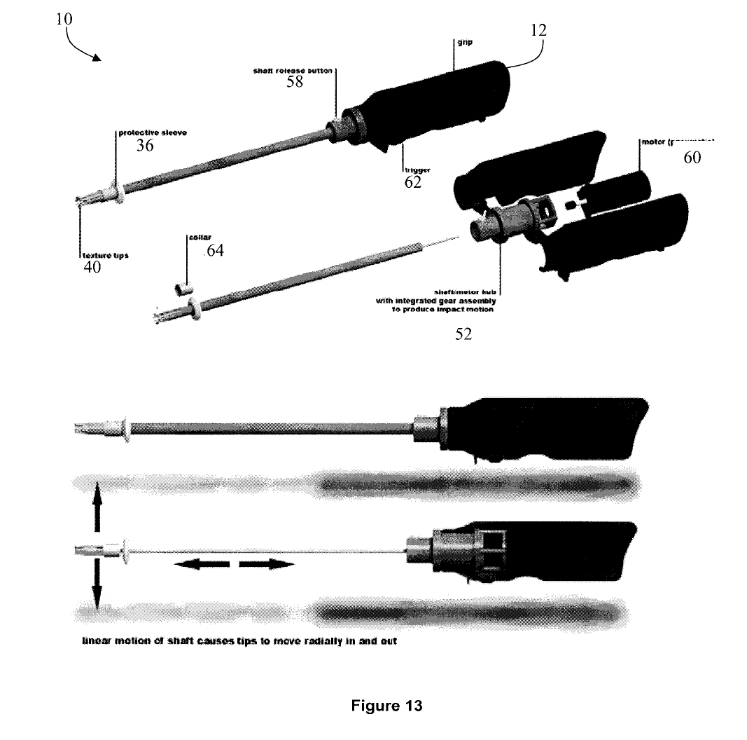

A fifth embodiment of the device 10 is illustrated in FIGS. 13 and 14. This device is similar to the embodiment of FIGS. 9 and 10 but differs in that instead of rotation of the elongate shaft 42, the elongate shaft 42 is made to move parallel to its long axis (in and out). This linear motion causes the fingers/tips to move radially in and out as the distal end of the elongate shaft 42 pushes the tips outwardly. The distal end of the elongate shaft is in the form of an arrow head or the like, which is tapered, such that movement of the elongate shaft horizontally to the axis of the elongate shaft 42 causes the taper or the arrow head to push against and withdraw from the fingers/tips so that they extend outwardly and retract. This is similar to the embodiment of the device of FIGS. 11 and 12. This results in all the tips/fingers moving outwardly and inwardly at the same time and therefore making impact with the bone surface at the same time. This device is particularly well suited to texturing the endosteal cortex.

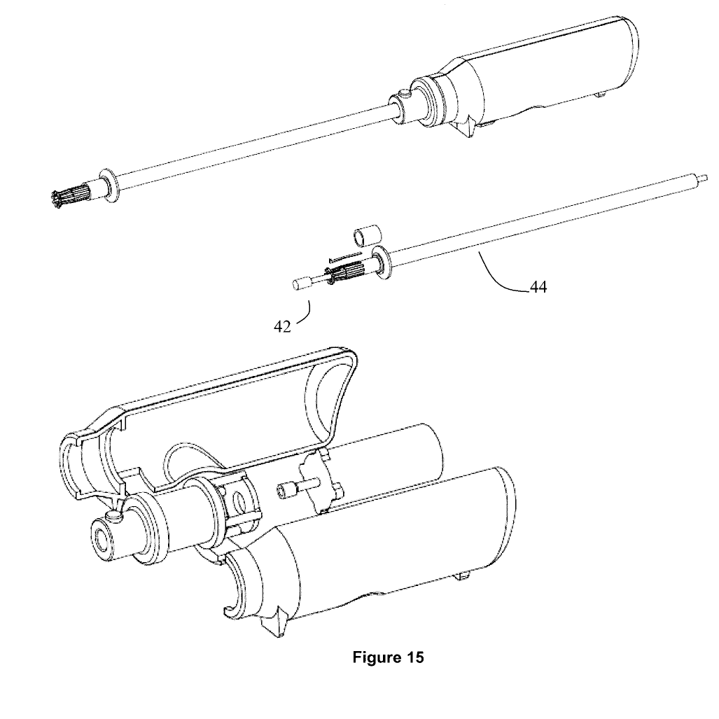

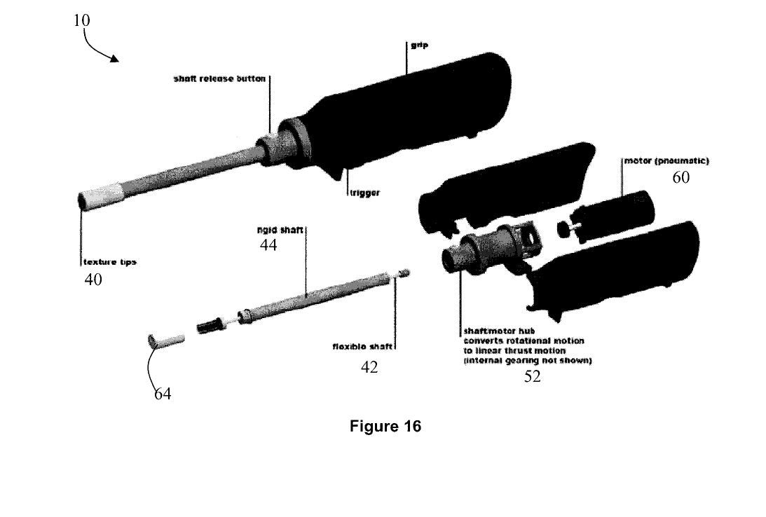

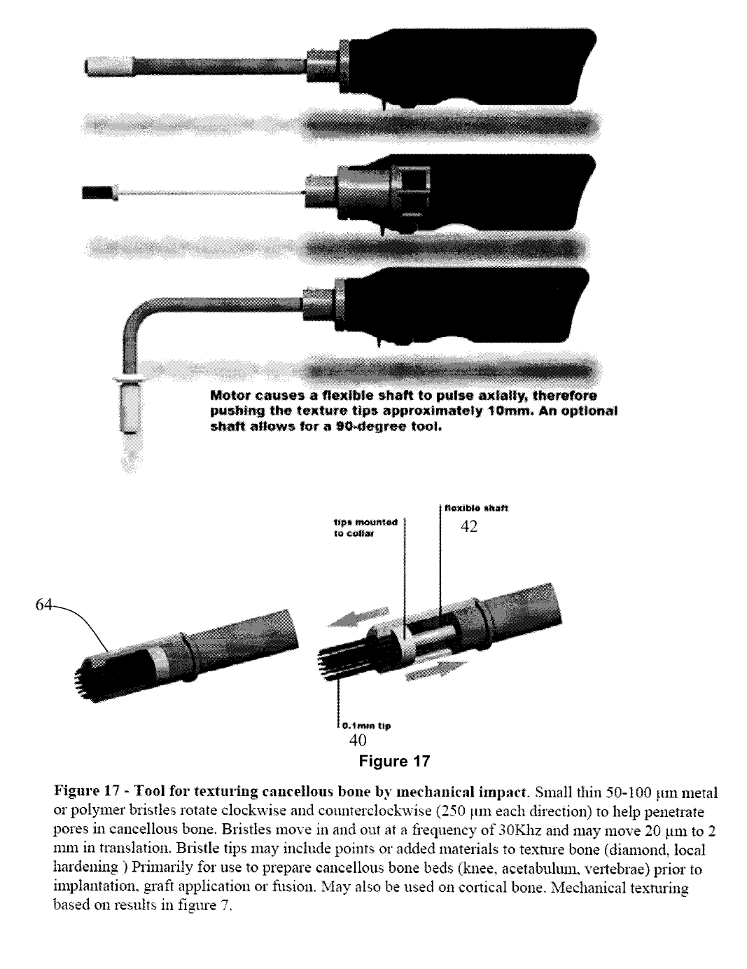

A sixth embodiment of the device is illustrated in FIGS. 16 to 18. This embodiment of the device 10 is particularly well suited to texturing cancellous bone or bone with very irregular surfaces. For example, this embodiment of the device 10 could be used to prepare cancellous bone beds (knee, acetabulum, vertebrae) prior to implantation, graft application or fusion. This embodiment differs from that of FIGS. 11 and 12 in that the elongate shaft 42 and the sleeve 44 are flexible. The sleeve is more rigid than the elongate shaft to protect it. The elongate shaft 42 or the sleeve 44 can be provided with a mechanism to lock them into position once bent to the desired shape i.e. to transition from flexible to rigid, in a manner known to persons skilled in the art. The impact surfaces 40 at the distal end of the elongate shaft 42 are in the form of a plurality of bristles/needles/fibres which each have a diameter of about 50 to 100 μm. The bristles are flexible. The bristles can be made of metal or a polymer or the like. The bristles may vibrate in many directions at both ultrasonic and non-ultrasonic frequencies. The fibres may move in micron and millimeter distances in all directions. There may be slight rotation clockwise or anticlockwise to aid ingress of the bristles.

The motor 60 imparts a linear motion to the elongate shaft which causes the bristles to extend in and out of the collar. The motion imparted to the elongate shaft is linear (parallel to the long axis of the shaft) which causes the bristles to move in and out of the collar 64. The bristles can move in and out at a frequency of 30 kHz and may move 20 μm to 10 mm in translation. Alternatively, the bristles may move in and out of the collar out at one frequency and amplitude, and may vibrate at another frequency and amplitude. In this respect, the motor may move the bristles in and out in the 1-10 mm range. Another motor may vibrate the bristles near their base to move them in the 10-50 μm range at about 30 kHz. The elongate shaft may be angled, such as by 90°, for ease of accessibility to the bone surface to be textured. The bristle tips may include points or added materials to alter their surface roughness and mechanical properties such as diamond or local hardening. This embodiment of the device 10 can help to texture bone without fracturing it. It can also be used to texture cortical bone within the endosteal canal.

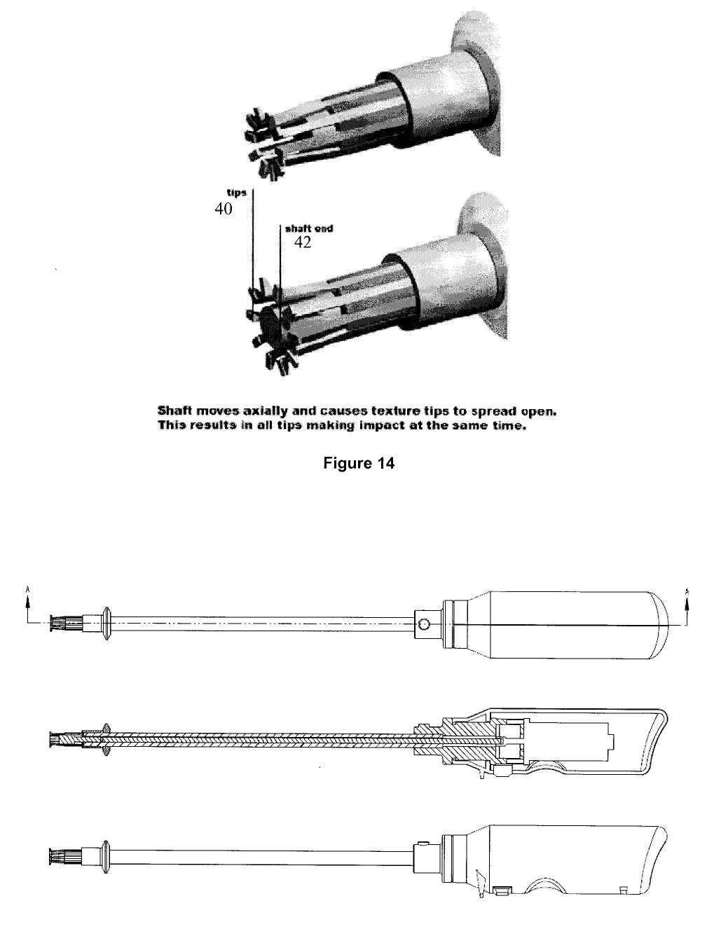

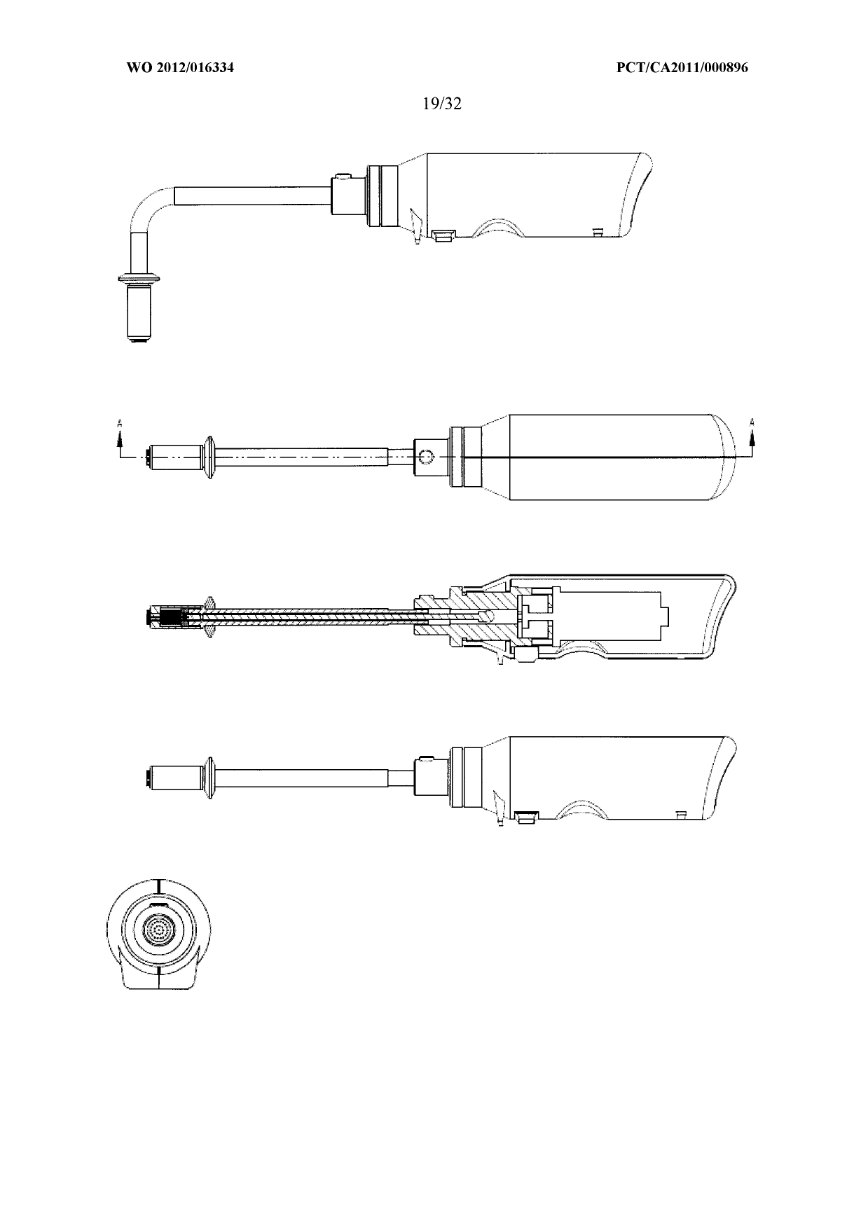

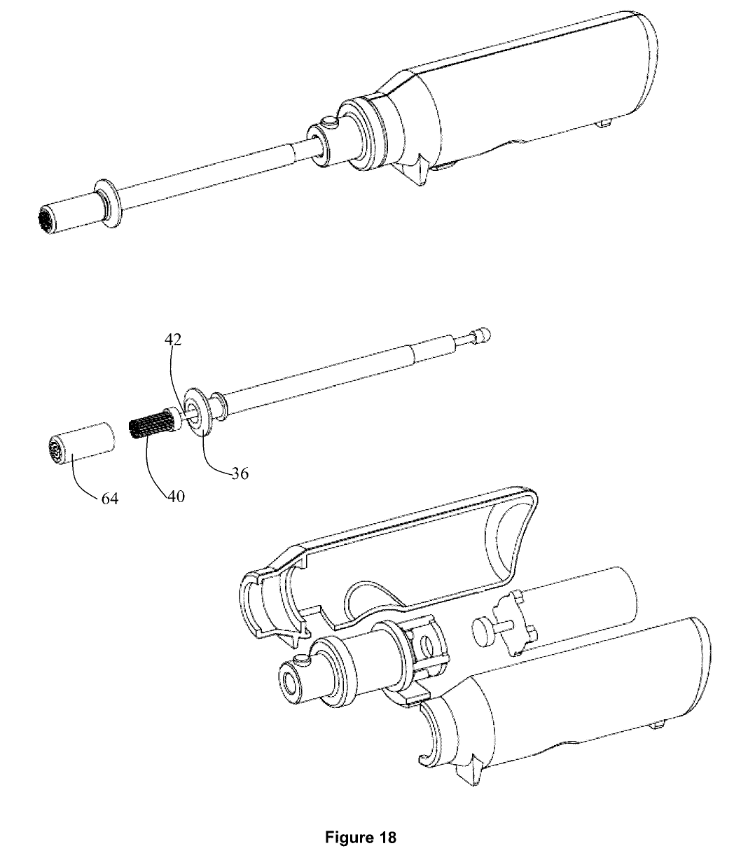

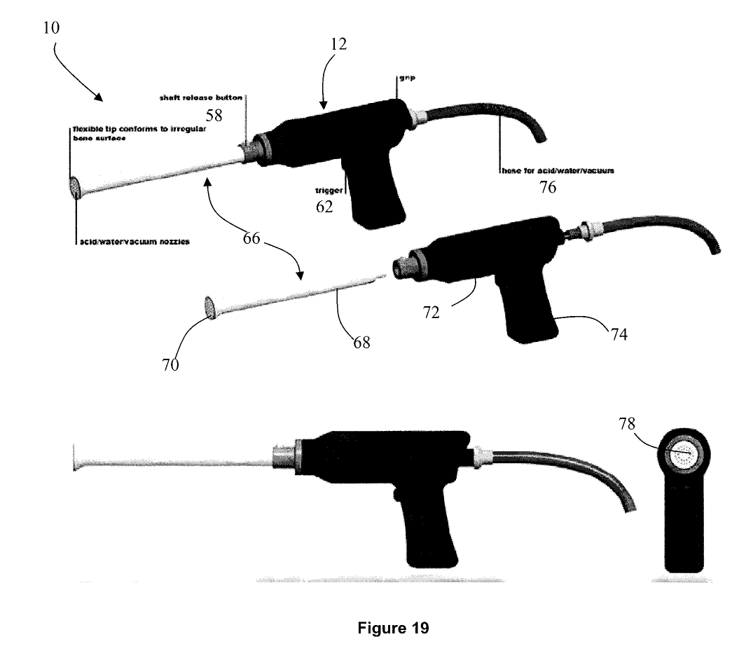

A seventh embodiment of the device is illustrated in FIGS. 19 to 21. This embodiment of the device differs from those of FIGS. 1 to 18, in that instead of texturing a bone surface using mechanical means, it is arranged to deliver a solution to a bone surface to roughen the bone surface, such as by etching or the like. This device is particularly well suited to preparing cancellous bone beds, such as on the knee, acetabulum and vertebrae, prior to implantation, graft application or fusion. The device may also be used on cortical bone. The solution to be delivered by the device can be phosphoric acid (15%) or any other suitable organic or inorganic acid or any other etching solution. The device is also arranged remove fluids from the bone surface.

In this embodiment of the device 10, instead of an impact tip, there is provided a nozzle 66 having an elongate hollow body 68 and a distal nozzle tip 70 for delivery of fluids to a bone surface and for evacuation of the fluids from the bone surface. The nozzle 66 can be removeably attached to the handle 12. In this respect, the button 58 at the distal end of the handle can release the nozzle 66 from the handle 12 and attach the nozzle 66 to the handle 12. The handle 12 is in the form of a pistol for ease of handling, although any other suitable shape or form is also possible. The handle 12 includes a hollow body portion 72 through which the fluids will flow and a grip portion 74 which a user holds which may also include an internal mechanism (not shown) for evacuating the fluid in and out of the nozzle 66 and the trigger 62 for controlling the flow of the fluids. The body portion 72 of the handle 12 includes an opening for receiving a hose 76 through which the fluids are delivered from a container (not shown).

The nozzle tip 70 comprises an array of openings 78. In the illustrated embodiment, the array comprises three concentric circles of openings 78, although the array may vary from this configuration. As illustrated, the innermost circle of openings is for the delivery of the etching solution, the middle circle of openings is for the delivery of water and the outermost circle of openings is for the removal of the fluids such as by applying a vacuum or a suction force to the bone surface. It is thought that delivery and collection of the fluids being via different openings 78 will enhance the circulation of the fluid through the bone. Alternatively, all the openings may be used for the delivery of all the fluids.

In use, the nozzle tip 70 is held against the bone surface to be textured. The etching fluid is applied through the openings 78, such as by pulling the trigger 62. Optionally, the etching fluid is then removed by applying a vacuum away from the bone or sucking the fluid away from the bone. Next, water is applied to the bone surface through the openings. Finally, the fluid (etching fluid and/or water) is removed by applying a vacuum away from the bone surface. The exposure to the etching fluid is preferably limited to the outermost 2 to 5 mm of bone. In this respect, a ‘barrier’ material, such as a gel, may be injected first into the bone to restrict the penetration of the etching fluid and form a barrier between the bone marrow and the etching fluid. Application of the etching fluid may push the gel back 1-2 mm and activate gelation. For example, a gel which sets at reduced pH can be used when the etching solution is an acid. The gel is then removed from the bone with the water rinse and vacuum steps, or with a vacuum step alone.

Preferably, one etching step is followed by 1 to 3 water rinse steps. Each etching step may last 1-5 seconds, and each rinsing step may last 5-10 seconds. The delivery of the etching solution or the water, or both, may be pulsatile to enhance penetration of the fluid into the bone. Ultrasonic motion may also be applied to aid the etching or the washing or both. Optionally, if the etching solution is an acid, an alkaline solution may be delivered to the bone surface to neutralize the acid, or vice versa.

Etching solutions which can be used include 15% phosphoric acid or 20% acetic acid for 15 seconds to 5 minutes, more preferably 30 seconds to 2 minutes, or any other suitable time. Other acids and combinations of acids may also provide a suitable etching effect. Other concentrations of acids are also possible, as will be clear to those skilled in the art.

The trigger 62 may control the operation of all the steps. Alternatively, different triggers may be provided for the application of the different fluids and for the initiation of the vacuum. The different steps may be automated and so one pull of the trigger initiates and ends the whole cycle.

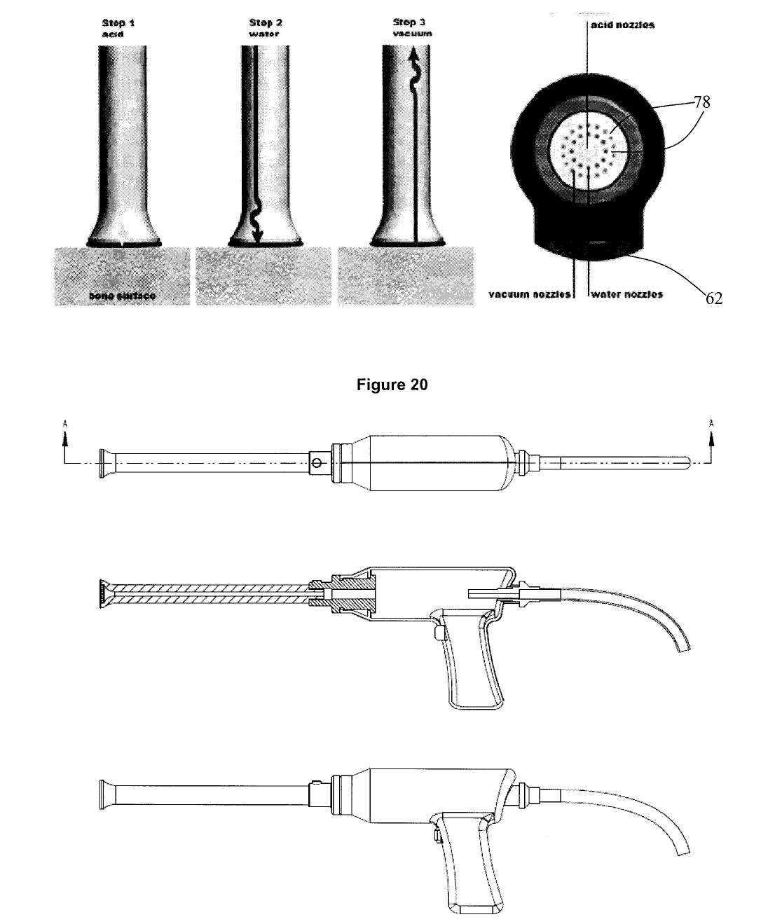

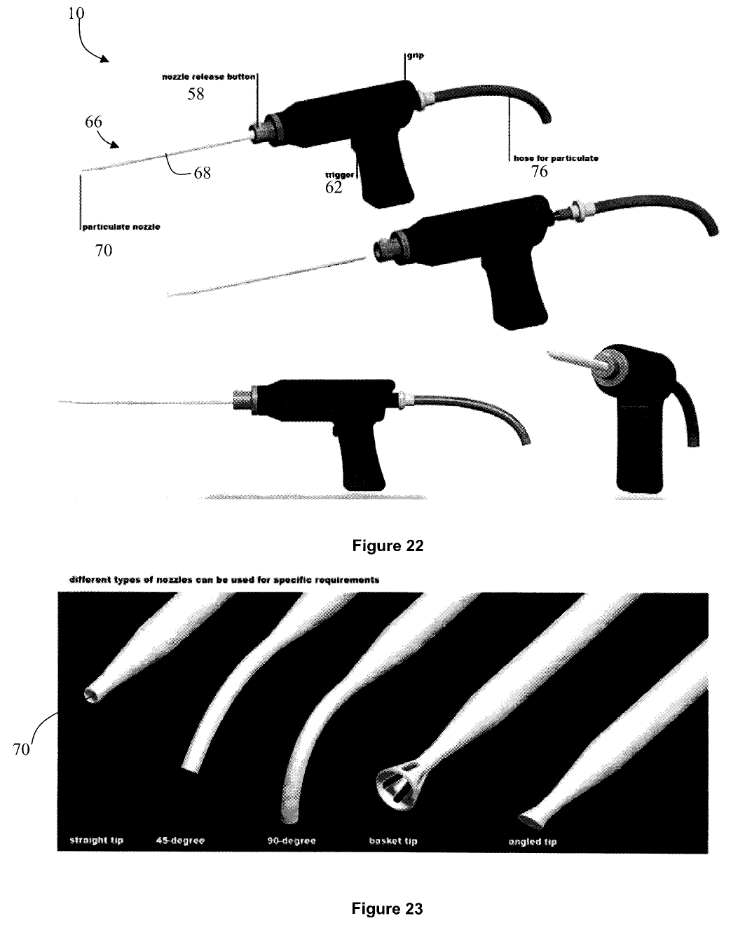

An eighth embodiment of the device is illustrated in FIGS. 22 to 24, and differs from the embodiment of FIGS. 19 to 21 in that the device is arranged to inject particles into cancellous bone or onto allografts. The particles provide a microtexture to the bone surface and are thought to stimulate bone formation. This device is intended for the preparation of cancellous bone beds such as knee, acetabulum and vertebrae prior to implantation, graft application or fusion. The particles may be crushed particles of bone retrieved and processed from the patient or another donor. The particles may be bioactive particles such as calcium phosphate compounds, hydroxyapatite compounds, alumina or the like. The particles may be inert such as silica or the like. The particles may be soluble such as salt or sugar or the like. The particle diameters may range from 1 to 1000 μm in diameter, preferably 10-500 μm, and more preferably 10-50 μm. The particles can be used to blast the bone surface before being substantially removed to provide the appropriate surface roughness or the particles can be arranged to be embedded in the bone surface. The particles are suspended in a fluid such as saline or air before being delivered to the bone surface. For roughening allografts, the slurry of particles are delivered under pressure such as 10-80 PSI and the nozzle tip 70 is held about 0.25 to 2 inches away from the bone surface. The impact tip can also be pressed against the bone surface. For injecting particles into a cancellous space, air is preferably used as a carrier as it can escape without entering the blood stream. These particles are preferably 5-10 μm in diameter.

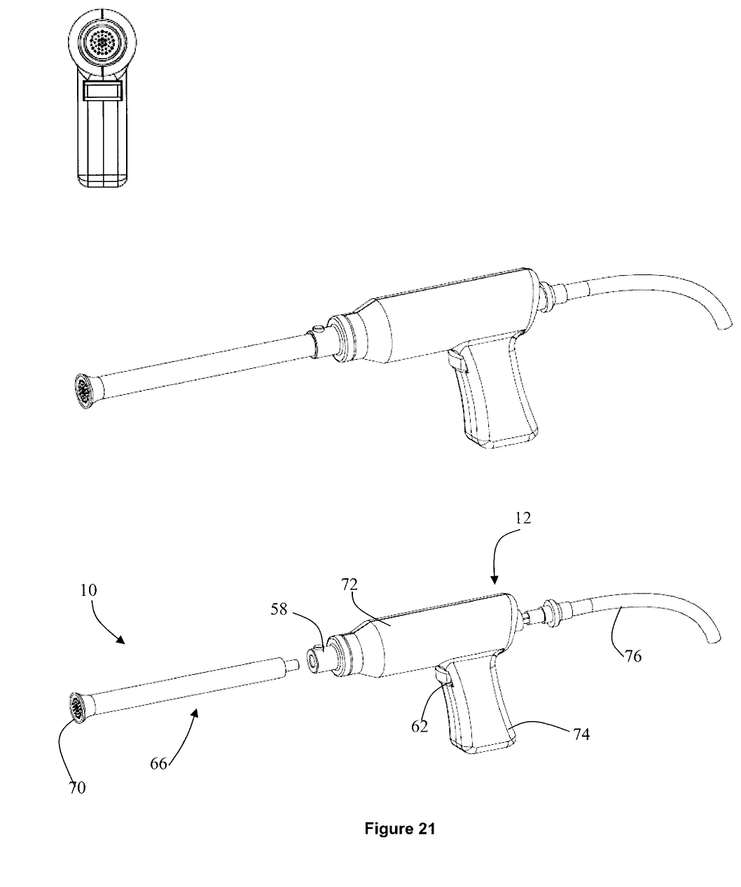

Therefore, instead of a nozzle tip 70 having a plurality of openings 78, the nozzle 66 has a single, larger opening 78 for delivering particles. As best seen in FIG. 23, the nozzle tip 70 may have different configurations such as a straight tip, a tip angled to the nozzle body 68, a basket tip and an angled tip.

It will be appreciated that the bone surface may be etched before or after treatment with the particles.

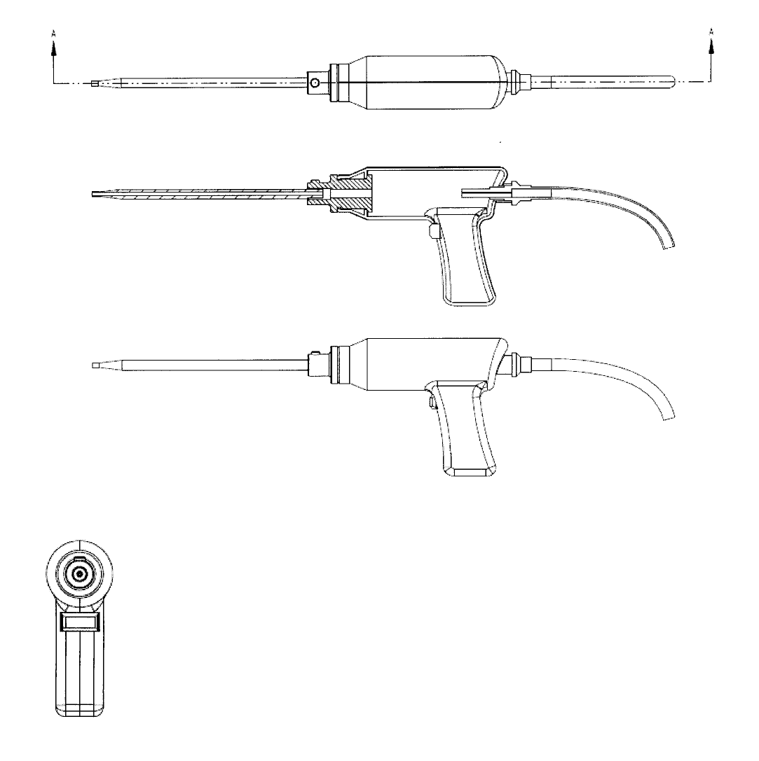

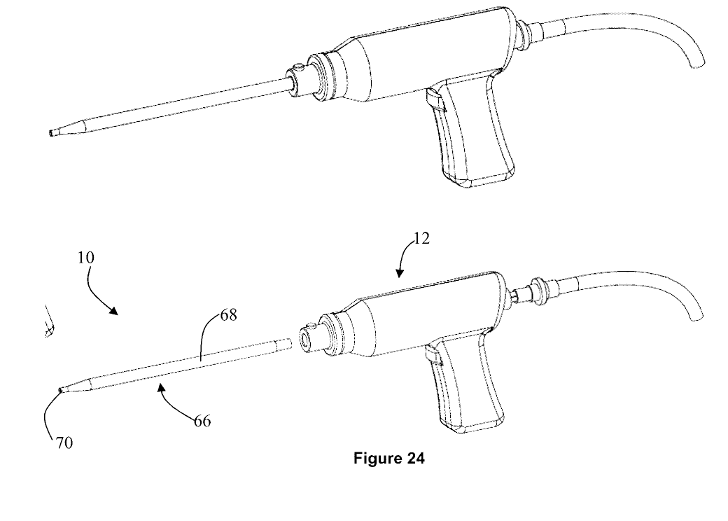

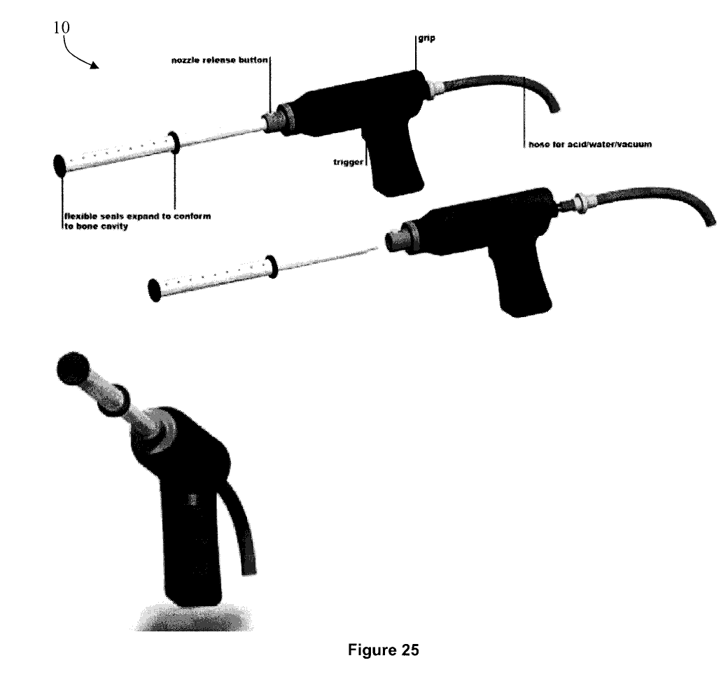

An eighth embodiment of the device is illustrated in FIGS. 25 to 27, and differs from the embodiment of FIGS. 19 to 21 in that the device is arranged to etch endosteal bone. In this respect, instead of the nozzle 66 having openings 78 at a distal end, the openings 78 are provided along at least a portion of the length of the elongate body 68 of the nozzle 66 and the distal end is sealed. The nozzle body includes seals 80 delineating the portion of the length having the openings so that the area of bone exposed to the etching fluid can be limited. The nozzle body 68 is arranged to be received into a bone cavity where an etching fluid can be delivered. In use, the seals 80 separate the portion of the bone to be treated from the portion of the bone to be left untreated.

From another aspect, there is provided a method of dissolution of a bone surface to apply a surface texture similar to that of a fracture surface of bone.

In one embodiment, the method comprises soaking the bone, such as an allograft, in an acid bath, for example 15% phosphoric acid or 20% acetic acid for about 1-5 minutes or any other suitable time such as 30 seconds to 2 minutes. Other acids and combinations may also work. Vacuum, sonication, forming channels through the bone, stirring, heating, cooling and movement of the bone graft or the bath, and combinations thereof, may be employed to enhance penetration into porous material, reduce etch time and preserve as much of the bone mass as possible. For example, channels formed in the bone can help to get the acid quickly to the interior and sonication can help to degass and move the acid in and out. Also, an alternating vacuum (positive pressure and negative pressure) or a vacuum alone, can also be used to drive fluid through the bone block.

There is also provided a carrier such as a sponge, brush or a gel to apply acid to a bone surface or to insert into the medullary cavity of bone.

From another aspect, there is provided a method of adding material to a bone surface to adapt that bone surface to be similar to a fracture surface of bone. The material can be particulates or precipitates.

For particulates, crushed cancellous bone can be used to create particles with many fractured surfaces. Particulates are 0.1-20 μm in diameter, ideally 1-5 μm. A thin slurry is formed using a binder that can dry and harden or can be removed at temperatures that will not damage the bone. Polyethylene glycol is one example. Polycaprolactone (PCL) can also be used as a binder, diluted in a solvent such as tetrahydrofuran (THF). THF is allowed to evaporate leaving PCL and bone on surface. CaP can also be used as a particulate.

The bone surface may also be adapted by immersion in a solution that promotes precipitation or crystallization of calcium and phosphate or similar Ca or P compounds or other biologically relevant compounds on the bone surface. Crystallisation may be enhanced by first etching the surface to provide sites to initiate crystallisation. To enhance precipitation, bone can also be lightly etched to provide nucleation sites. Bone is soaked in solution of Ca(OH)₂ at 28° C., add H₃PO₄ until a white precipitate forms. Bone is heated to 100° C. to fuse the precipitate to the bone surface and to itself. Heating may also favorably change the surface morphology of the precipitate.

### Example 3

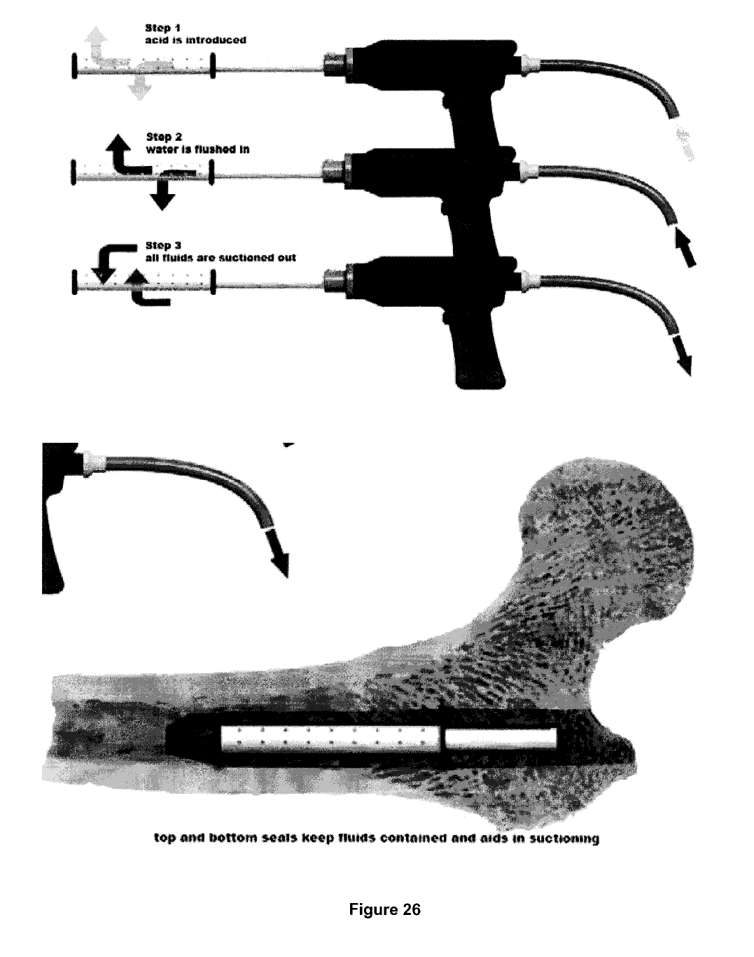

FIG. 28 illustrates polished cortical bone disks textured by abrasive blasting at a blast pressure of about 80 PSI, a nozzle distance of about 15 cm and Al₂O₃ particles of size about 100-200 μm. Particles were suspended in air. FIG. 28A: Low magnification (25×) SEM image of a polished bone disk with textured and untextured areas. FIG. 28B: Low magnification (100×) SEM image of the textured and untextured interface. FIG. 28C: Higher magnification (500×) image of the bone surface textured by abrasive blasting. FIG. 28D: High power image (1000×) of polished bone (control) surface, double the magnification of C. Blast media can also be salt, sugar or CaP compounds, ice crystals, dry ice, or bone particulates or other compounds that can be dissolved after blasting.

### Example 4

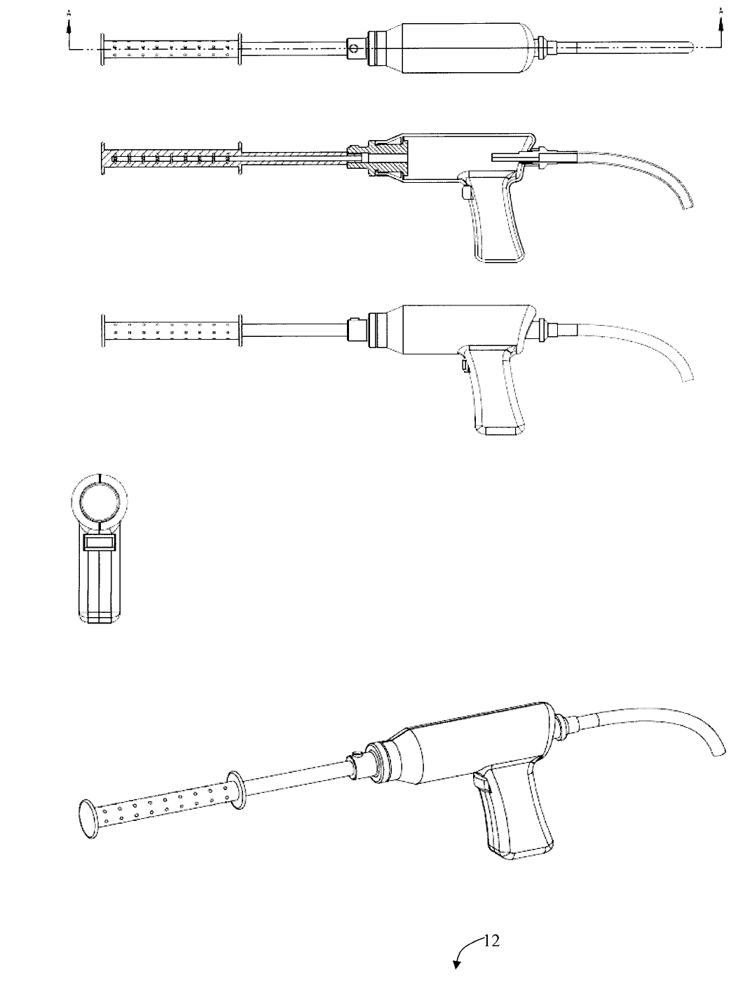

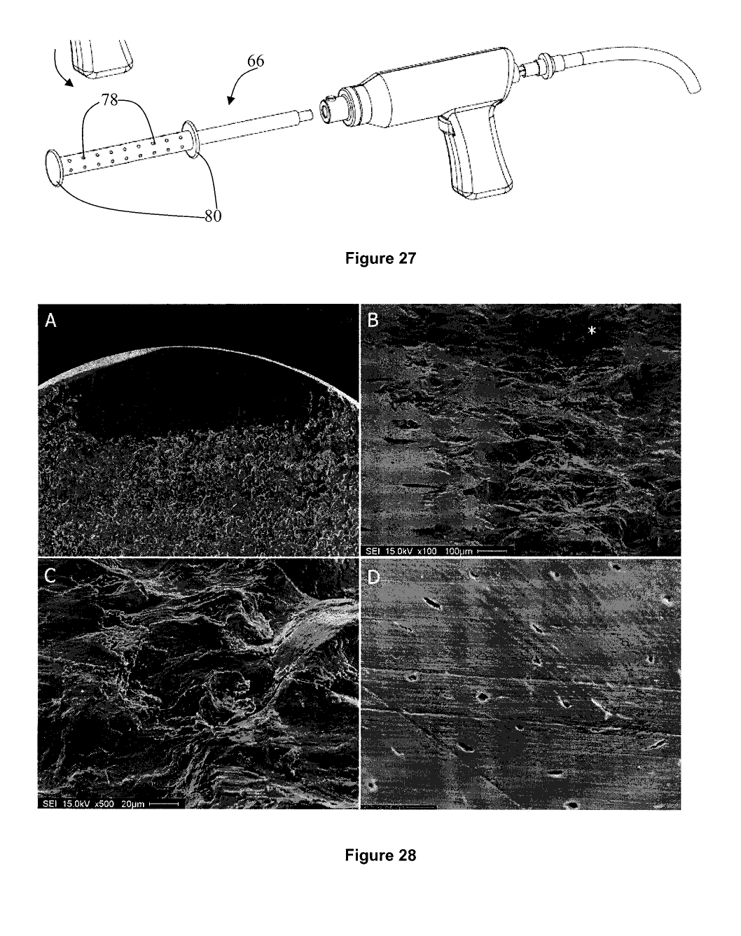

FIG. 29 illustrates SEM images of polished bone disks after being exposed to phosphoric acid (H₃PO₄) (15%) for 2 minutes. FIG. 29A: Medium power image demonstrating micro and macro texturing. FIG. 29B: Higher power image demonstrating complex porous surface with a homogeneous texture. FIG. 29C: High power image shows submicron sized features (Rₐ˜1.5 μm). FIG. 29D: High power image of polished bone surface equal in magnification to B. Acid etching can be used as a secondary treatment in addition to abrasive (particulate) texturing, or alone. Can be used as a bath soak for allograft, cortical and cancellous bone, optionally with sonication and heat to aid acid activity. FIG. 30 is an SEM image (43×) of cancellous bone etched with phosphoric acid (H₃PO₄) (15%) for 2 minutes.

### Example 5

### Precipitation of Ca Compounds on Bone Surfaces

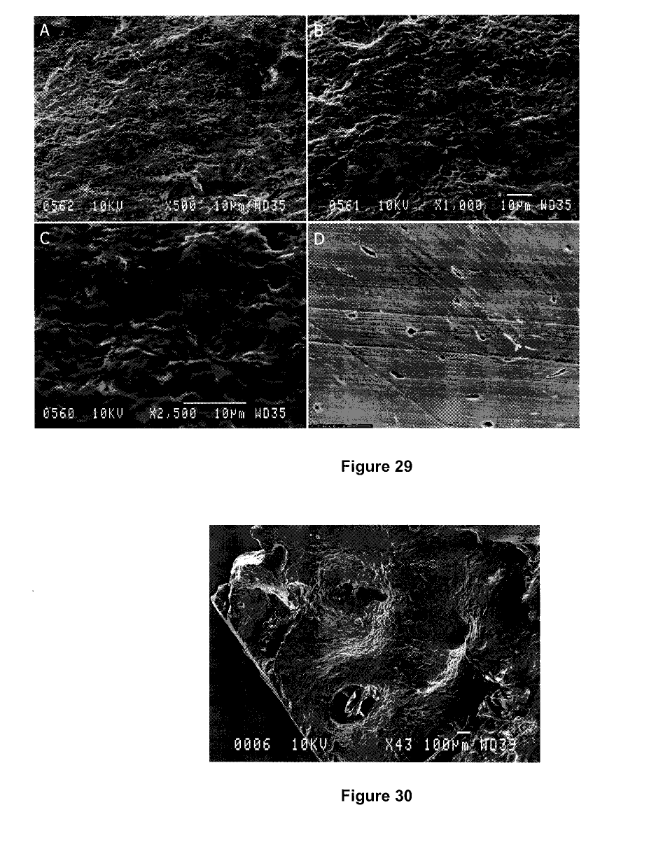

FIG. 31 shows SEM images of cancellous bone which has been immersed in supersaturated CaCO₃ at boiling point, cooled to room temperature, then heated to 250° F. for 10 minutes. FIG. 31A: Low power image of untreated (control cancellous bone) with characteristic ‘smooth’ surfaces (Rₐ<0.5 μm). Note that outermost surfaces have been cut and as a result present a texture. FIG. 31B: Low power SEM image of cancellous bone treated by immersion in a supersaturated CaCO₃ solution. Significant roughness is apparent. FIG. 31C: Formation of crystal structures are visible on the cancellous bone which are more evident at higher magnification FIG. 31D. (Rₐ estimated˜5-7 μm). This method can also be used as a secondary treatment in addition to abrasive (particulate) texturing or alone. Can be used as a bath soak for allograft, cortical and cancellous bone, with sonication and heat to aid penetration. Other precipitation methods are possible such as Sol gel for HA etc.

### Example 6

### Binding of Ca Based Particulates

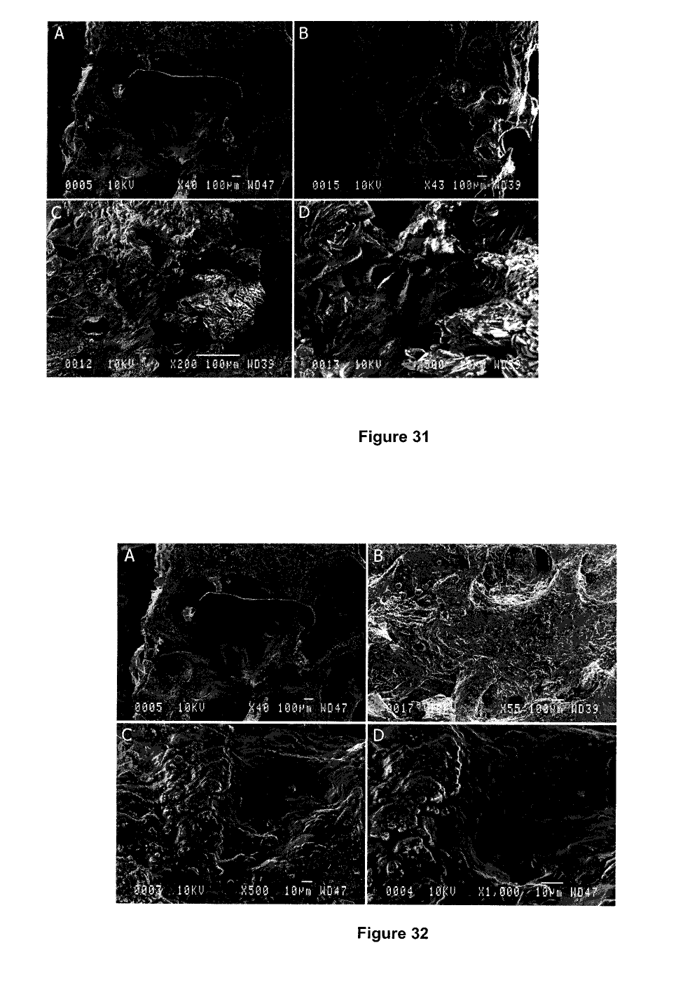

FIG. 32 shows SEM images of cancellous bone immersed in CaCl₂ suspension, withdrawn then heated to 250° F. for 10 minutes. FIG. 32A: Low power (40×) image of untreated (control cancellous bone) with characteristic ‘smooth’ surfaces (Rₐ<0.5 μm). Note that outermost surfaces have been cut and as a result present a texture. FIG. 32B: Low power (43×) SEM image of cancellous bone treated by immersion in a suspension of CaCl₂. FIG. 32C (500×): CaCl₂ particles are visible as a coating on the bone surface. CaCl₂ suspension forms a surface with considerable macro and micro roughness. FIG. 32D: (1000×) Submicron sized features are visible at higher power. (Rₐ estimated˜3-5 μm). Can be used as a secondary treatment in addition to abrasive (particulate) texturing. Bath soak for allograft, cortical and cancellous bone, with sonication and heat to aid penetration. Other coating methods: HA powders, bone particulate, binders such as polycaprolactone etc.

It should be appreciated that the invention is not limited to the particular embodiments described and illustrated herein but includes all modifications and variations falling within the scope of the invention as defined in the appended claims.

1. A device for imparting a surface roughness to bone, the device comprising:
a body having an impacting tip at a distal end, the body or the impacting tip being removeably attachable to a handle;
a means for generating reciprocating motion of the impacting tip, wherein the impacting tip is arranged to impart a surface roughness similar to that of a bone fracture surface.

2. (canceled)

3. A device according to claim 1 wherein the impacting tip comprises a blade having a cutting edge extending from a blade holder.

4. A device according to claim 1, wherein the direction of the reciprocating movement is in the same direction as a long axis of the body.

5. A device according to claim 1, wherein the means for generating the reciprocating motion is selected from the group consisting of a pneumatic, electric, magnetorestrictive, or piezo electric device.

6. A device according to claim 1, further comprising a tool for attaching and detaching the impacting tip to the body, the tool having a first end for receiving the impacting tip and attaching it to the body, and a second end for receiving the impacting when attached to the body and detaching it from the body.

7. A device according to claim 1, further comprising a spacer for spacing the impacting tip from the bone surface to be impacted, the spacer extending alongside the impacting tip and being attached to a part of the device which does not reciprocate.

8. A device according to claim 1, wherein the means for generating the reciprocating motion is at least partially in the handle.

9. A device according to claim 1, further comprising a sleeve surrounding the body along at least a portion of its length.

10. (canceled)

11. (canceled)

12. A device according to claim 9, further comprising a spacer for spacing the impacting tip from the bone surface to be impacted, the spacer being annular and being positioned on the sleeve.

13. (canceled)

14. A device according to claim 1, wherein the means for generating a reciprocating motion imparts a motion of the impacting tip substantially perpendicular to a long axis of the body.

15. A device according to claim 1, wherein the impacting tip is at least one impacting surface.

16. A device according to claim 15, wherein the impacting tip comprises two impacting surfaces angled with respect to one another.

17. A device according to claim 9, further comprising an adjustment sleeve positioned around the sleeve and moveable with respect to the sleeve for adjusting the amplitude of motion of the impacting tip.

18. A device according to claim 1, wherein the impacting tip comprises a plurality of arms which are moveable with respect to the body, the arms being arranged to move outwardly when the body moves.

19. A device according to claim 18, wherein each arm has a bone contacting surface which may be angled relative to the arm.

20. (canceled)

21. (canceled)

22. (canceled)

23. A device according to claim 1, wherein the impacting tip comprises blades arranged around a distal end of the body and moveable with respect to the body.

24. (canceled)

25. (canceled)

26. A device according to claim 23, further comprising a cap positionable at the distal end of the body over the blades, and having openings through which the blades extend as the body actuates towards and away from the blades.

27. A device according to according to claim 1, wherein the impacting tip comprises fibres attached to a distal end of the body.

28. A device according to claim 27, further comprising a collar attached to the sleeve at the distal end of the body, such that movement of the body causes the fibres to move in and out of the collar.

29. A device according to claim 27, wherein the means for generating a reciprocating motion imparts a linear motion of the body towards and away from the fibres.

30. A device according to claim 27, wherein the body is flexible such that it can be angled relative to the handle.

31. A device for imparting a surface roughness to bone, the device comprising:
a hollow body having a delivery tip at a distal end, the body being removeably attachable to a handle;
a means for supplying fluid to and from the delivery tip to impart a surface roughness similar to that of a bone fracture surface.

32. (canceled)

33. A device according to claim 31, wherein the fluid being delivered to and from the delivery tip includes an etching fluid and water.

34. (canceled)

35. A device according to claim 31, wherein the delivery tip is angled relative to the body.

36. A device according to claim 31, wherein the delivery tip comprises a series of openings arranged along a portion of a length of the body, the distal end of the body being sealed.

37. A device according to claim 36, further comprising an annular seal along the body inbetween the portion of the body with the openings and a portion of the body without the openings.

38. A device according to claim 31, further comprising a memory for storing a cycle of fluid delivery and fluid suction through the delivery tip.

39. (canceled)

40. (canceled)

41. A method for imparting a surface roughness to bone comprising impacting a surface of bone with the impacting tip of the device of claim 1.

42. A method for imparting a surface roughness to bone comprising etching a surface of bone using the device of claim 31.

43. (canceled)

44. (canceled)

45. (canceled)
